SEQUENCE LISTING (20 sequences)

SEQ ID NO: 1 - DNA, 549 bp, synthetic construct
  source 1..549: mol_type = other DNA
atgggcggaggagcccctacccaggacgagttcacccagctgagccagagcatcgctgagttccacacctaccagctgggaaacggacgctgttccagcctgctggcacagagaatccacgctcctcctgagacagtgtggagtgtggtgcgcagattcgaccgccctcagatttacaagcacttcatcaagagctgcaacgtgagcgaggacttcgagatgagagtgggatgtaccagagatgtgaacgtgatcagcggactgcctgccaacaccagcagagagagactggacctgctggacgatgaccgcagagtgaccggcttcagcatcaccggaggtgagcacagactgagaaactacaagagcgtgaccaccgtccaccgcttcgagaaggaagaggaagaggagcgcatctggaccgtggtgctggagagctacgtcgtggacgtgcccgagggcaacagcgaagaggatacccgcctgttcgctgacaccgtgatcagactgaacctccagaagctggccagcatcaccgaggcaatgaac

SEQ ID NO: 2 - protein, 183 aa, synthetic construct
MGGGAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 3 - protein, 177 aa, synthetic construct
TQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 4 - skipped

SEQ ID NO: 5 - DNA, 902 bp, synthetic construct
  source 1..902: mol_type = other DNA
tgaccagagtgcccctgtacggcttcaccagcatttgtggcagacggcccgaaatggaagccgccgtgtctacaatccccagattcctccagagcagcagcggctccatgctggacggcagattcgatcctcagagcgccgctcacttcttcggcgtgtacgatggacatggcggaagccaggtggccaactactgccgcgaaagaatgcatctggccctggccgaggaaatcgccaaagaaaagcccatgctgtgcgacggcgacacctggctggaaaagtggaagaaggccctgttcaacagcttcctgagagtggacagcgagatcgagagcgtggcccctgaaacagtgggcagcacatctgtggtggccgtggtgtttcccagccacatcttcgtggctaactgcggcgatagcagagccgtgctgtgcagaggaaaaacagccctgcctctgtccgtggaccacaagcctgatagagaggatgaggccgccagaattgaagccgctggcggcaaagtgatccagtggaatggcgctagagtgttcggcgtgctggccatgagtagatccatcggcgatagatacctgaagcctagcatcatccccgatcctgaagtgaccgccgtgaagagagtgaaagaggacgactgcctgatcctggcctctgacggtgtctgggacgtgatgacagatgaagaggcctgcgagatggcccggaagagaatcctgctgtggcacaagaaaaacgccgtggccggggatgcttctctgctggctgacgagagaagaaaagagggcaaagaccccgctgccatgtctgccgccgagtacctgtctaagctggccatccagagaggcagcaaggacaacatcagcgtggtggtcgtggacctgaaa

SEQ ID NO: 6 - protein, 301 aa, synthetic construct
MTRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLK

SEQ ID NO: 7 - protein, 298 aa, synthetic construct
VPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLK

SEQ ID NO: 8 - skipped

SEQ ID NO: 9 - DNA, 45 bp, synthetic construct
  source 1..45: mol_type = other DNA
actagtggcggcggaggcagcggaggcggatctggcggaggatct

SEQ ID NO: 10 - protein, 15 aa, synthetic construct
TSGGGGSGGGSGGGS

SEQ ID NO: 11 - DNA, 45 bp, synthetic construct
  source 1..45: mol_type = other DNA
acgcgtggcggcggaggcagcggaggcggatctggcggaggatct

SEQ ID NO: 12 - protein, 15 aa, synthetic construct
TRGGGGSGGGSGGGS

SEQ ID NO: 13 - DNA, 45 bp, synthetic construct
  source 1..45: mol_type = other DNA
ggcggcggaggaagtggcggcggatctggcggaggatctaccggt

SEQ ID NO: 14 - protein, 15 aa, synthetic construct
GGGGSGGGSGGGSTG

SEQ ID NO: 15 - protein, 23 aa, synthetic construct
DQSNSEEAKKEEAKKEEAKKSNS

SEQ ID NO: 16 - protein, 40 aa, synthetic construct
SGGGSGGGSGGGSGGSGGSGGGSGGSGGSGGGSGGGSGGG

SEQ ID NO: 17 - protein, 12 aa, synthetic construct
ESKYGPPAPPAP

SEQ ID NO: 18 - protein, 49 aa, Synthetic construct
AVGQDTQEVIVVPHSLPFKVVVISAILALVVLTIISLIILIMLWQKKPR

SEQ ID NO: 19 - DNA, 147 bp, Synthetic construct
  source 1..147: mol_type = other DNA
gccgtcggccaggacacccaagaagtgatcgtcgtccctcacagcctgcctttcaaggtggtggtcatcagcgccattctggccctggtggtgctgaccatcatcagcctgatcatcctgattatgctgtggcagaagaagcccaga

SEQ ID NO: 20 - protein, 75 aa, synthetic construct
  VARIANT 1..75: note = GGGGS repeats may vary between 1-15. Up to 14 GGGGS repeats may be absent.
GGGGSGGGGSGGGGSGGGGSGGGGSGGGGSGGGGSGGGGSGGGGSGGGGSGGGGSGGGGSGGGGSGGGGSGGGGS

(19) United States
(12) Patent Application Publication
(10) Pub. No.: US 2025/0313815 A1
(43) Pub. Date: Oct. 9, 2025

(54) ACTIVE LOADING OF CARGO ENTITY INTO LIPID BILAYER PARTICLES USING DIMERIZATION DOMAINS

(71) Applicant: Northwestern University, Evanston, IL (US)

(72) Inventors: Joshua N. Leonard, Evanston, IL (US); Devin Stranford, Evanston, IL (US)

(73) Assignee: Northwestern University, Evanston, IL (US)

(21) Appl. No.: 18/865,216

(22) PCT Filed: May 13, 2023
(86) PCT No.: PCT/US2023/022168

§ 371 Date: Nov. 12, 2024

Related U.S. Application Data
(60) Provisional application No. 63/341,906, filed on May 13, 2022.

(51) Int. Cl. C12N 9/16; A61K 9/127; A61K 9/50

(52) U.S. Cl. CPC C12N 9/16; A61K 9/127; A61K 9/5068; C07K 2319/03; C07K 2319/055; C12Y 301/03016

(57) ABSTRACT

The present disclosure relates generally to methods and compositions for loading cargo entities into lipid bilayer particles, such as cell-derived membrane particles, e.g., secreted extracellular vesicles.

### CROSS-REFERENCE TO RELATED APPLICATION

This application is the U.S. National Stage of PCT/US2023/022168, which claims priority to U.S. provisional application No. 63/341,906, filed on May 13, 2022, which is incorporated herein by reference in its entirety.

The instant application contains a Sequence Listing which has been submitted in XML format and is hereby incorporated by reference in its entirety. Said XML copy, created on Jul. 13, 2023, is named 121384-0203.xml and is 20,480 bytes.

### STATEMENT OF GOVERNMENT SUPPORT

This invention was made with government support under Grant No. P30 All 17943 awarded by the National Institutes of Health. The United States government has certain rights in the invention.

### TECHNICAL FIELD

The present disclosure relates generally to methods and compositions for loading cargo entities into secreted lipid bilayer particles (e.g., cell-derived membrane particles such as extracellular vesicles).

### BACKGROUND

The following discussion is merely provided to aid the reader in understanding the disclosure and is not admitted to describe or constitute prior art thereto.

Secreted extracellular vesicles (EVs), such as exosomes and microvesicles, are nanometer-scale lipid vesicles that are produced by many cell types and transfer proteins, nucleic acids, and other entities between cells in the human body, as well as those of other animals. EVs have a wide variety of potential therapeutic uses and are an attractive platform for delivering a wide variety of therapeutics. For example, targeted exosomes have already been shown to be effective for delivery of RNA to neural cells and tumor cells in mice. Other cell-derived membrane particles can also be used for similar purposes.

Protein cargo can be loaded into membrane particles by mass action through overexpression in EV producer cells in a method often referred to as “passive loading.” However, passive loading is inefficient, particularly for large protein cargo and for certain cell-derived membrane particles or vesicles. In addition, there is a need for engineering multifunctional vesicles, e.g., EVs including more than one enriched protein cargo. The disclosed technology aims to address these limitations of the current technologies.

### SUMMARY OF THE PRESENT TECHNOLOGY

The present disclosure provides chimeric peptides, as well as systems and methods for using the same, for the loading of cargo entities (e.g., a nucleic acid cargo, polypeptide cargo, a nucleocapsid cargo, and combinations thereof) into lipid bilayer particles (e.g., cell-derived membrane particles (CDMPs), including but not limited to, extracellular vesicles.

In one aspect, the present disclosure provides chimeric proteins or peptides comprising a cargo-loading domain comprising an abscisic acid-insensitive 1 (ABI1) sequence. In some embodiments, chimeric proteins or peptides is directly or indirectly linked to a cargo entity.

In one aspect, the present disclosure provides chimeric proteins or peptides comprising: (a) a cargo entity; and (b) a cargo-loading domain comprising an abscisic acid-insensitive 1 (ABI1) sequence. In some embodiments, the chimeric proteins or peptides may further comprise a linker that connects the cargo entity and the cargo-loading domain. In some embodiments, the linker may comprise:
(1) an amino acid sequence selected from SEQ ID NO: 10 (TSGGGGSGGGSGGGS), SEQ ID NO: 12 (TRGGGGSGGGSGGGS), SEQ ID NO: 14 (GGGGSGGGSGGGSTG), SEQ ID NO: 15 (DQSNSEEAKKEEAKKEEAKKSNS), SEQ ID NO: 16 (SGGGSGGGSGGGSGGSGGSGGGSGGSGGSGGGSGGGSGGG), and SEQ ID NO: 17 (ESKYGPPAPPAP); or
(2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 10, 12, 14, 15, 16, or 17.

In some embodiments, the cargo-loading domain is a truncated variant of a wild-type protein that comprises an extracellular vesicle targeting domain. In some embodiments, the cargo-loading domain comprises residues 126-423 of wild type ABI1. In some embodiments, the cargo-loading domain comprises:

(SEQ ID NO: 6)
MTRVPLYGFTSICGRRPEMEAAVSTIPRFLQSSSGSMLDGRFDPQSAAH
FFGVYDGHGGSQVANYCRERMHLALAEEIAKEKPMLCDGDTWLEKWKKA
LFNSFLRVDSEIESVAPETVGSTSVVAVVFPSHIFVANCGDSRAVLCRG
KTALPLSVDHKPDREDEAARIEAAGGKVIQWNGARVFGVLAMSRSIGDR
YLKPSIIPDPEVTAVKRVKEDDCLILASDGVWDVMTDEEACEMARKRIL
LWHKKNAVAGDASLLADERRKEGKDPAAMSAAEYLSKLAIQRGSKDNIS
VVVVDLK,
(SEQ ID NO: 7)
VPLYGFTSICGRRPEMEAAVSTIPRFLQSSSGSMLDGRFDPQSAAHFFG
VYDGHGGSQVANYCRERMHLALAEEIAKEKPMLCDGDTWLEKWKKALFN
SFLRVDSEIESVAPETVGSTSVVAVVFPSHIFVANCGDSRAVLCRGKTA
LPLSVDHKPDREDEAARIEAAGGKVIQWNGARVFGVLAMSRSIGDRYLK
PSIIPDPEVTAVKRVKEDDCLILASDGVWDVMTDEEACEMARKRILLWH
KKNAVAGDASLLADERRKEGKDPAAMSAAEYLSKLAIQRGSKDNISVVV
VDLK,

a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 6 or 7, or
a functional fragment of SEQ ID NO: 6, SEQ ID NO: 7, or a variant amino acid sequence thereof.

In some embodiments, the cargo entity is a cytosolic cargo entity. In some embodiments, the cargo entities is a membrane-bound cargo entity.

In another aspect, the present disclosure also provides lipid bilayer particle (e.g., cell-derived membrane particle (CDMP)) loading systems comprising a chimeric protein or peptide as disclosed here (e.g., any of the foregoing aspects or embodiments) and a second chimeric protein or peptide comprising (i) a second cargo molecule, and (ii) and membrane-bound domain comprising an abscisic acid (ABA)-binding sequence, wherein the second chimeric protein or peptide optionally comprises a second linker that connects the second cargo entity and the ABA-binding sequence.

In some embodiments, the ABA-binding sequence comprises a pyrabactin resistance 1-like (PYL1) sequence. In some embodiments, the PYL1 sequence comprises residues 33-209 of wild type PYL1. In some embodiments, the PYL1 sequence comprises

(SEQ ID NO: 2)
MGGGAPTQDEFTQLSQSIAEFHTYQLGNGRCSSLLAQRIHAPPETVWSV
VRRFDRPQIYKHFIKSCNVSEDFEMRVGCTRDVNVISGLPANTSRERLD
LLDDDRRVTGFSITGGEHRLRNYKSVTTVHRFEKEEEEERIWTVVLESY
VVDVPEGNSEEDTRLFADTVIRLNLQKLASITEAMN,
(SEQ ID NO: 3)
TQDEFTQLSQSIAEFHTYQLGNGRCSSLLAQRIHAPPETVWSVVRRFDR
PQIYKHFIKSCNVSEDFEMRVGCTRDVNVISGLPANTSRERLDLLDDDR
RVTGFSITGGEHRLRNYKSVTTVHRFEKEEEEERIWTVVLESYVVDVPE
GNSEEDTRLFADTVIRLNLQKLASITEAMN,

or
a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 2 or 3, or
a functional fragment of SEQ ID NO: 6, SEQ ID NO: 7, or a variant amino acid sequence thereof.

In some embodiments, the second linker comprises:
(1) an amino acid sequence selected from SEQ ID NO: 10, SEQ ID NO: 12, SEQ ID NO: 14, SEQ ID NO: 15, SEQ ID NO: 16, and SEQ ID NO: 17; or
(2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 10, 12, 14, 15, 16, or 17.

In some embodiments, a disclosed lipid bilayer particle (e.g., CDMP) loading system may further comprises abscisic acid (ABA).

In some embodiments, the second cargo entity is a membrane-bound cargo molecule, wherein the cargo entity optionally comprises (i) a targeting protein and (ii) a transmembrane domain, and wherein the targeting protein is selected from an antibody, a Fab, a Fab′, a F(ab′)2, a Fd, a scFv, a single-chain antibody, a disulfide-linked Fvs (sdFv), a de novo-designed binding molecule, an affinibody, a DARPIN, and a nanobody.

In another aspect, the present disclosure provides lipid bilayer particle (e.g., cell-derived membrane particles (CDMP)) comprising a chimeric protein or peptide as disclosed herein (e.g., any one of the foregoing aspects or embodiments) or a lipid bilayer particle (e.g., CDMP) loading system as disclosed herein (e.g., any one of the foregoing aspects or embodiments). In some embodiments, lipid bilayer particles (e.g., CDMPs) are selected from extracellular vesicles, virus particles, virus-like particles (VLPs), apoptotic bodies, platelet-like particles, and combinations thereof. In some embodiments, CDMPs are extracellular vesicles selected from the group consisting of exosomes, microvesicles, and combinations thereof.

In another aspect, the present disclosure provides nucleic acid encoding any one of the chimeric proteins or peptides disclosed herein or any one of the lipid bilayer particle (e.g., CDMP) loading system as disclosed herein. For example, in some embodiments, the cargo-loading domain of the chimeric protein or peptide is encoded by

(SEQ ID NO: 5)
ATGACCAGAGTGCCCCTGTACGGCTTCACCAGCATTTGTGGCAGACGGC
CCGAAATGGAAGCCGCCGTGTCTACAATCCCCAGATTCCTCCAGAGCAG
CAGCGGCTCCATGCTGGACGGCAGATTCGATCCTCAGAGCGCCGCTCAC
TTCTTCGGCGTGTACGATGGACATGGCGGAAGCCAGGTGGCCAACTACT
GCCGCGAAAGAATGCATCTGGCCCTGGCCGAGGAAATCGCCAAAGAAAA
GCCCATGCTGTGCGACGGCGACACCTGGCTGGAAAAGTGGAAGAAGGCC
CTGTTCAACAGCTTCCTGAGAGTGGACAGCGAGATCGAGAGCGTGGCCC
CTGAAACAGTGGGCAGCACATCTGTGGTGGCCGTGGTGTTTCCCAGCCA
CATCTTCGTGGCTAACTGCGGCGATAGCAGAGCCGTGCTGTGCAGAGGA
AAAACAGCCCTGCCTCTGTCCGTGGACCACAAGCCTGATAGAGAGGATG
AGGCCGCCAGAATTGAAGCCGCTGGCGGCAAAGTGATCCAGTGGAATGG
CGCTAGAGTGTTCGGCGTGCTGGCCATGAGTAGATCCATCGGCGATAGA
TACCTGAAGCCTAGCATCATCCCCGATCCTGAAGTGACCGCCGTGAAGA
GAGTGAAAGAGGACGACTGCCTGATCCTGGCCTCTGACGGTGTCTGGGA
CGTGATGACAGATGAAGAGGCCTGCGAGATGGCCCGGAAGAGAATCCTG
CTGTGGCACAAGAAAAACGCCGTGGCCGGGGATGCTTCTCTGCTGGCTG
ACGAGAGAAGAAAAGAGGGCAAAGACCCCGCTGCCATGTCTGCCGCCGA
GTACCTGTCTAAGCTGGCCATCCAGAGAGGCAGCAAGGACAACATCAGC
GTGGTGGTCGTGGACCTGAAA,

a variant nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 5, or
a functional fragment of SEQ ID NO: 5 or a variant nucleic acid sequence thereof. In some embodiments, the ABA-binding sequence of the second chimeric protein or peptide is encoded by

(SEQ ID NO: 1)
ATGGGCGGAGGAGCCCCTACCCAGGACGAGTTCACCCAGCTGAGCCAGA
GCATCGCTGAGTTCCACACCTACCAGCTGGGAAACGGACGCTGTTCCAG
CCTGCTGGCACAGAGAATCCACGCTCCTCCTGAGACAGTGTGGAGTGTG
GTGCGCAGATTCGACCGCCCTCAGATTTACAAGCACTTCATCAAGAGCT
GCAACGTGAGCGAGGACTTCGAGATGAGAGTGGGATGTACCAGAGATGT
GAACGTGATCAGCGGACTGCCTGCCAACACCAGCAGAGAGAGACTGGAC
CTGCTGGACGATGACCGCAGAGTGACCGGCTTCAGCATCACCGGAGGTG
AGCACAGACTGAGAAACTACAAGAGCGTGACCACCGTCCACCGCTTCGA
GAAGGAAGAGGAAGAGGAGCGCATCTGGACCGTGGTGCTGGAGAGCTAC
GTCGTGGACGTGCCCGAGGGCAACAGCGAAGAGGATACCCGCCTGTTCG
CTGACACCGTGATCAGACTGAACCTCCAGAAGCTGGCCAGCATCACCGA
GGCAATGAAC,

a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, or
a functional fragment of SEQ ID NO: 1 or a variant nucleic acid sequence thereof. In some embodiments, the linker and/or second linker are encoded by one of SEQ ID NOs: 9 (ACTAGTGGCGGCGGAGGCAGCGGAGGCGGATCTGGCGGAGGATCT), 11 (ACGCGTGGCGGCGGAGGCAGCGGAGGCGGATCTGGCGGAGGATCT), or 13 (GGCGGCGGAGGAAGTGGCGGCGGATCTGGCGGAGGATCTACCGGT),
or a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to one of SEQ ID NOs: 9, 11, or 13.

In another aspect, the present disclosure provides cells comprising a chimeric protein or peptide as disclosed herein, a lipid bilayer particles (e.g., CDMP) loading system as disclosed herein, a lipid bilayer particle (e.g., CDMP) as disclosed here, or a nucleic acid as disclosed herein. In some embodiments, the cell is a mammalian cell, wherein the mammalian cell is optionally selected from HEK293, HEK293FT, a mesenchymal stem cell, a megakaryocyte, an induced pluripotent stem cell (iPSC), a T cell, an erythrocyte, an erythropoetic precursor, and an iPSC-derived version of any of the preceding cells.

In another aspect, the present disclosure provides methods of loading a cargo entity into lipid bilayer particle (e.g., CDMP), comprising expressing in a cell a chimeric protein or peptide as disclosed herein. In some embodiments, loading of the cargo entity of the chimeric protein or peptide is enhanced compared to passive cargo loading. In some embodiments, the cargo entity is a a viral nucleocapsid, a synthetic nucleic acid, transcription factor, a recombinase, a base editor, a prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, or a ribonucleoprotein complex.

In another aspect, the present disclosure provides methods of loading two cargo entities into lipid bilayer particle (e.g., cell-derived membrane particle (CDMP), comprising expressing in a cell the lipid bilayer particle (e.g., CDMP) loading system as described herein. In some embodiments, co-localization of the cargo entity of the chimeric protein or peptide and the second cargo entity of the second chimeric protein or peptide is enhanced compared to passive cargo loading. In some embodiments, the cargo entity is a viral nucleocapsid, a synthetic nucleic acid, a transcription factor, a recombinase, a base editor, a prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, or a ribonucleoprotein complex.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIGS. 1A-1G show that cargo protein is actively loaded into EVs via tagging with the ABI domain of the abscisic acid dimerization system. FIG. 1A. Illustration of abscisic acid-based dimerization of EV cargo proteins and subsequent loading into vesicles. FIG. 1B. ABA-induced dimerization between PYL and ABI domains. Illustrative microscopy showing scFv-PYL (membrane bound) and EYFP-ABI (cytosolic) association in the presence of ABA. Full images are in FIGS. 4A-4B. FIG. 1C. ABI-induced cargo loading into EVs. EVs generated under conditions indicated were adsorbed to aldehyde/sulfate latex beads and analyzed by flow cytometry to determine bulk average fluorescence. Experiments were performed in biological triplicate, and error bars indicate standard error of the mean. Statistical tests comprise two-tailed Student's t-tests using the Benjamini-Hochberg method to reduce the false discovery rate (*p<0.05, **p<0.01, ***p<0.001). FIG. 1D. Representative histograms of EYFP+/−ABI conditions in FIG. 1C. FIG. 1E. Active loading of Cas9-ABI with and without an NLS into EVs. 6.0×10⁸ EVs were loaded per lane. Expected band sizes (˜160 or 195 kDa, arrows) correspond to Cas9−/+ the ABI domain. The full blot is provided in FIG. 6D. FIG. 1F. Analysis of ABA-dependent Cas9-ABI loading into EVs enriched for scFv-PYL via affinity chromatography. 1.3×10⁷ MVs or 2.0×10⁷ exosomes were loaded per lane. Expected band size: 195 kDa (arrows). Full blots are provided in FIG. 7B. FIG. 1G. Bioactivity of EV-associated Cas9. Vesicles were lysed and incubated with a linearized target plasmid for 1 h at 37° C. in Cas9 nuclease reaction buffer. Expected cut band sizes: 7.6 and 4.6 kb (arrows).

FIGS. 2A-3C show that EVs harvested via differential ultracentrifugation display characteristic surface markers, size distribution, and morphology. FIG. 2A. Detection of CD9 (25 kDa), CD81 (26 kDa), and Alix (96 kDa) in both microvesicle (MV) and exosome (Exo) EV fractions. EV fractions contained minimal calnexin (˜90 kDa). Expected band positions are indicated by arrows. 3 μg cell lysate or 4.5×10⁸ vesicles were loaded per lane. FIG. 2B. Representative NTA size distributions of EV subpopulations. Numbers above histograms refer to the mode size. Error bars (black) indicate standard error of the mean, calculated for each bin.

FIG. 2C. Representative TEM of EV subpopulations.

FIGS. 3A-3C show that ABA-binding domains can be incorporated into EV cargo proteins. FIG. 3A. Expression of EYFP fused to the ABI and PYL ABA-binding domains with and without an NLS in transiently transfected HEK293FT cells analyzed by flow cytometry. Experiments were performed in biological triplicate, and error bars indicate standard error of the mean. Statistical tests comprise two-tailed Student's t-tests using the Benjamini-Hochberg method to reduce the false discovery rate (*p<0.05, **p<0.01, ***p<0.001). FIG. 3B. Surface stain (via 3×FLAG tag) of HEK293FTs transfected with scFv constructs fused to ABI or PYL at the C-terminus. FIG. 3C. Expression of scFv constructs from FIG. 3B. 2 μg cell lysate was loaded per lane. Expected band sizes: ˜40, 62, and 75 kDa (arrows).

FIGS. 4A-4B show that ABA induces dimerization between the ABI and PYL domains. FIGS. 4A-4B HEK293FT cells transfected with scFv-PYL and EYFP-ABI were treated with EtOH (FIG. 4A) or ABA (FIG. 4B) and imaged via confocal microscopy. Brightfield, fluorescence, contrast-adjusted and pseudo-colored fluorescence, and overlays are shown.

FIGS. 5A-5C show that the ABI domain increases EV cargo loading independent of total protein expression. FIG. 5A. Expression of EYFP and EYFP-ABI in the presence of targeting constructs in transiently transfected HEK293FT cells was analyzed by flow cytometry. A key observation is that addition of the ABI domain does not increase overall cargo protein expression in producer cells. FIG. 5B. Repeat of EYFP-ABI EV loading trends in the presence of an scFv shown in FIG. 1C. FIG. 5C. Comparison of EYFP loading into EVs with and without an NLS with ABA-binding constructs and under ABA-induced dimerization conditions. Addition of an NLS did not substantially impact EYFP loading, nor did ABA-induced dimerization substantially impact loading of nuclear-localized cargo. Experiments were performed in biological triplicate, and error bars indicate standard error of the mean. Statistical tests comprise two-tailed Student's t-tests using the Benjamini-Hochberg method to reduce the false discovery rate (*p<0.05, **p<0.01, ***p<0.001).

FIGS. 6A-6E show that the ABI domain increases Cas9 loading into EVs and Cas9-ABI retains function. FIG. 6A. Expression of Cas9 fused to either the ABI or PYL domain in transiently transfected HEK293FT cells. 2 μg cell lysate was loaded per lane. Expected band sizes: ˜160, 183, and 195 kDa (arrows). FIG. 6B. Cartoon illustrating the Cas9 reporter construct. Successful editing by Cas9 results in the deletion of a stop codon and (in some random fraction of cases) a repair-mediated frame shift induces express dTomato. FIG. 6C. Absence of an NLS or presence of the ABI domain does not meaningfully reduce Cas9 editing efficiency in transiently transfected Jurkat T cells. Cells were analyzed by flow cytometry 3 d post-transfection. Experiments were performed in biological triplicate, and error bars indicate standard error of the mean. Statistical tests comprise two-tailed Student's t-tests using the Benjamini-Hochberg method to reduce the false discovery rate (*p<0.05, **p<0.01, ***p<0.001). Samples with high cellular autofluorescence were excluded from analysis. FIG. 6D. Full blot of Cas9 EV active loading data presented in FIG. 1E. FIG. 6E. Cellular expression of Cas9 with and without the ABI domain or an NLS. 2 μg cell lysate was loaded per lane.

FIG. 7 shows that EVs populations can be separated by affinity chromatography to analyze cargo loading patterns. FIG. 7A. Validation of affinity chromatography technique. 3×FLAG tagged scFv containing vesicles were run through an anti-FLAG affinity matrix and analyzed for the FLAG tag to demonstrate enrichment in the eluted population. 1.5×10⁷ EVs were loaded per lane. Expected band size: ˜62 kDa (arrow). FIG. 7B. Full blots of affinity-isolated EV Cas9 content with and without ABA-induced dimerization presented in FIG. 1F.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, microbiology and recombinant DNA are used. See, e.g., Sambrook and Russell eds. (2001)Molecular Cloning: A Laboratory Manual, 3rd edition; the series Ausubel et al., eds. (2007) Current Protocols in Molecular Biology; the series Methods in Enzymology (Academic Press, Inc., N.Y.); MacPherson et al., (1991) PCR 1: A Practical Approach (IRL Press at Oxford University Press); MacPherson et al., (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al., eds (1996) Weir's Handbook of Experimental Immunology.

The present disclosure is based, in part, on the discovery (1) that fusion of a truncated abscisic acid-insensitive 1 (ABI1) protein to a cargo protein, alone, increases loading of the cargo protein into lipid bilayer particles (e.g., cell-derived membrane particles such as extracellular vesicles or “EVs”), and (2) that the cargo and a membrane protein can be fused to domains that heterodimerize upon binding the small entity abscisic acid (ABA) such that, upon ABA addition, respective domains can dimerize, which may increase cargo loading into lipid bilayer particles (e.g., cell-derived membrane particles, such as EVs). ABA-mediated dimerization of ABI1 and PYL1 is reversible, thus the present technology would also allow cargo release, e.g., after a lipid bilayer particle (e.g., cell-derived membrane particle) fuses to a target cell and exposes the particle's interior to the recipient cell cytoplasm.

### Definitions

Unless otherwise specified or indicated by context, the terms “a”, “an”, and “the” mean “one or more.” For example, “a fusion protein,” “an extracellular vesicle,” and “a cell” should be interpreted to mean “one or more fusion proteins,” “one or more extracellular vesicles,” and “one or more cells,” respectively.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, a “control” is an alternative sample used in an experiment for comparison purpose. A control can be “positive” or “negative.” For example, where the purpose of the experiment is to determine a correlation of the efficacy of cargo protein loading to EVs and the structures of the cargo proteins, a positive control (a cargo protein known to exhibit the desired loading efficacy) and a negative control (a cargo protein that does not load to EVs) are typically employed.

As used herein, the term “extracellular vesicles” should be interpreted to include all nanometer-scale lipid vesicles that are secreted and/or budding by cells such as exosomes and microvesicles, respectively. As used herein, the term “exosomes” refer to extracellular vesicles originate from internal endocytic compartments and multi-vesicular bodies, and the term “microvesicles” refer to vesicles that bud directly from the cell surface. EVs, and their isolation and analysis are well-known to a skilled in the art. See, for example, Doyle et al., Cells 8(7): 727 (2019), which is incorporated herein by reference in its entirety. Extracellular vesicles may be taken up by so-called extracellular vesicle (EV) recipient cells. As utilized herein, the term “recipient cell” may be interchangeably with the term “target cell.”

As used herein, the term “engineered” refers to the aspect of having been designed, produced, and/or manipulated by the hand of man. For example, a polynucleotide is considered to be “engineered” when two or more sequences that are not linked together in that order in nature are designed or otherwise caused by the hand of man to be directly linked to one another in the engineered polynucleotide and/or when a particular residue in a polynucleotide is non-naturally occurring and/or is caused through action of the hand of man to be linked with an entity or moiety with which it is not linked in nature. For example, in some embodiments described and/or utilized herein, an engineered polynucleotide comprises a regulatory sequence that is found in nature in operative association with a first coding sequence but not in operative association with a second coding sequence, is linked by the hand of man so that it is operatively associated with the second coding sequence. Comparably, in some embodiments a polypeptide may be considered to be “engineered” if encoded by or expressed from an engineered polynucleotide, and/or if produced other than natural expression in a cell. Analogously, a cell or organism is considered to be “engineered” if it has been subjected to a manipulation, so that its genetic, epigenetic, and/or phenotypic identity is altered relative to an appropriate reference cell such as otherwise identical cell that has not been so manipulated. In some embodiments, the manipulation is or comprises a genetic manipulation, so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). In some embodiments, an engineered cell is one that has been manipulated so that it contains and/or expresses a particular agent of interest (e.g., a protein, a nucleic acid, and/or a particular form thereof) in an altered amount and/or according to altered timing relative to such an appropriate reference cell. As is common practice and is understood by those in the art, progeny of an engineered polynucleotide or cell are typically still referred to as “engineered” even though the actual manipulation was performed on a prior entity.

As used herein “engineered lipid bilayer particles” refers to a lipid bilayer particle engineered as described herein. For example, in some embodiments, a lipid bilayer particle may be considered to be “engineered” if it is synthetically produced, i.e., not produced by a cell. Alternatively or additionally, in some embodiments, a lipid bilayer particle may be considered to be “engineered’ if it is produced by an engineered production cell. In some embodiments, an engineered lipid bilayer particle is produced by a production cell engineered to have a first chimeric protein comprising a cargo-loading domain and optionally a second chimeric protein. In some such embodiments, an engineered production cell differs from an appropriate reference cell in that it has been engineered to express a first chimeric protein comprising a cargo-loading domain, a second chimeric protein as described herein, or both, or to express one or both at a different level (e.g., an elevated level) such that lipid bilayer particles (e.g., CDMPs) produced (e.g., released) by such engineered production comprises significantly more cargo entities than comparable particles produced (e.g., released) by the reference cell.

As used herein, the term “cell-derived membrane particle” should be interpreted to include any membrane-derived vesicles or particle that can be generated by blebbing or budding, and can include hybrid vesicles generated by mixing vesicles that were generated from cells and synthetic vesicles, as well as vesicles or particles generated by mechanically processing cells. Thus, “cell-derived membrane particles” can include, but is not limited to, extracellular vesicles (as defined above), virus particles, virus-like particles (VLPs), apoptotic bodies, and platelet-like particles, and combinations thereof.

As used herein, the term “gene” means a segment of DNA that contains all the information for the regulated biosynthesis of an RNA product, including promoters, exons, introns, and other untranslated regions that control expression.

As used herein, “homology” or “identity” or “similarity” refers to sequence similarity between two peptides or between two nucleic acid entities. Homology can be determined by comparing a position in each sequence which may be aligned for purposes of comparison. When a position in the compared sequence is occupied by the same base or amino acid, then the entities are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences,” FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. Coli, plants, and other host cells.

Regarding polynucleotide sequences, a “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

“Transformation” or “transfected” describes a process by which exogenous nucleic acid (e.g., DNA or RNA) is introduced into a recipient cell. Transformation or transfection may occur under natural or artificial conditions according to various methods well-known in the art and may rely on any known method for the insertion of foreign nucleic acid sequences into a prokaryotic or eukaryotic host cell. The method for transformation or transfection is selected based on the type of host cell being transformed and may include, but is not limited to, bacteriophage or viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g., in vitro or ex vivo administration) or target tissues (e.g., in vivo administration). The term “transformed cells” or “transfected cells” includes stably transformed or transfected cells in which the inserted DNA is capable of replication either as an autonomously replicating plasmid or as part of the host chromosome, as well as transiently transformed or transfected cells which express the inserted DNA or RNA for limited periods of time. In another embodiment, the term also includes stably transfected cells.

The polynucleotide sequences contemplated herein may be present in expression vectors. For example, the vectors may comprise: (a) a polynucleotide encoding an ORF of a cargo protein; and (b) a polynucleotide that expresses an ABA-binding domain, e.g., a pyrabactin resistance 1-like (PYL1) sequence or an abscisic acid-insensitive 1 (ABI1) sequence. The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

Any of the conventional vectors used for expression in eukaryotic cells may be used for directly introducing DNA into a subject. Expression vectors containing regulatory elements from eukaryotic viruses may be used in eukaryotic expression vectors (e.g., vectors containing SV40, CMV, or retroviral promoters or enhancers). Exemplary vectors include those that express proteins under the direction of such promoters as the SV40 early promoter, SV40 later promoter, metallothionein promoter, human cytomegalovirus promoter, murine mammary tumor virus promoter, and Rous sarcoma virus promoter. Expression vectors as contemplated herein may include eukaryotic or prokaryotic control sequences that modulate expression of a heterologous protein (e.g., the fusion protein disclosed herein). Prokaryotic expression control sequences may include constitutive or inducible promoters (e.g., T3, T7, Lac, trp, or phoA), ribosome binding sites, or transcription terminators.

The vectors contemplated herein may be introduced and propagated in a prokaryote, which may be used to amplify copies of a vector to be introduced into a eukaryotic cell or as an intermediate vector in the production of a vector to be introduced into a eukaryotic cell (e.g. Amplifying a plasmid as part of a viral vector packaging system). A prokaryote may be used to amplify copies of a vector and express one or more nucleic acids, such as to provide a source of one or more proteins for delivery to a host cell or host organism. Expression of proteins in prokaryotes may be performed using Escherichia coli with vectors containing constitutive or inducible promoters directing the expression of either a protein or a fusion protein comprising a protein or a fragment thereof. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; (iii) to aid in the purification of the recombinant protein by acting as a ligand in affinity purification (e.g., a His tag); (iv) to tag the recombinant protein for identification (e.g., such as Green fluorescence protein (GFP) or an antigen (e.g., HA) that can be recognized by a labelled antibody); (v) to promote localization of the recombinant protein to a specific area of the cell (e.g., where the protein is fused (e.g., at its N-terminus or C-terminus) to a nuclear localization signal (NLS) which may include the NLS of SV40, nucleoplasmin, C-myc, M9 domain of hnRNP A1, or a synthetic NLS). The importance of neutral and acidic amino acids in NLS have been studied. (See Makkerh et al. (1996) Curr Biol 6(8):1025-1027). Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

The presently disclosed methods may include delivering one or more polynucleotides, such as or one or more vectors as described herein, one or more transcripts thereof, and/or one or proteins transcribed therefrom, to a host cell. Further contemplated are host cells produced by such methods, and organisms (such as animals, plants, or fungi) comprising or produced from such cells. The disclosed extracellular vesicles may be prepared by introducing vectors that express mRNA encoding a fusion protein and a cargo RNA as disclosed herein. Conventional viral and non-viral based gene transfer methods can be used to introduce nucleic acids in mammalian cells or target tissues. Non-viral vector delivery systems include DNA plasmids, RNA (e.g., A transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell.

In the methods contemplated herein, a host cell may be transiently or non-transiently transfected (i.e., stably transduced) with one or more vectors described herein. In some embodiments, a cell is transfected as it naturally occurs in a subject (i.e., in situ). In some embodiments, a cell that is transfected is taken from a subject (i.e., explanted). In some embodiments, the cell is derived from cells taken from a subject, such as a cell line. Suitable cells may include stem cells (e.g., embryonic stem cells and pluripotent stem cells). A cell transfected with one or more vectors described herein may be used to establish a new cell line comprising one or more vector-derived sequences. In the methods contemplated herein, a cell may be transiently transfected with the components of a system as described herein (such as by transient transfection of one or more vectors, or transfection with RNA), and modified through the activity of a complex, in order to establish a new cell line comprising cells containing the modification but lacking any other exogenous sequence.

As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

Regarding proteins, the term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein entity having an amino acid sequence that differs from a reference protein or polypeptide entity. A variant or mutant may have one or more insertions, deletions, or substitutions of an amino acid residue relative to a reference entity. A variant or mutant may include a fragment of a reference entity. For example, a mutant or variant entity may one or more insertions, deletions, or substitution of at least one amino acid residue relative to a reference polypeptide.

Regarding proteins, a “deletion” refers to a change in the amino acid sequence that results in the absence of one or more amino acid residues. A deletion removes at least 1, 2, 3, 4, 5, 10, 20, 50, 100, or 200 amino acids residues or a range of amino acid residues bounded by any of these values (e.g., a deletion of 5-10 amino acids). A deletion may include an internal deletion or a terminal deletion (e.g., an N-terminal truncation or a C-terminal truncation of a reference polypeptide). A “variant,” “mutant,” or “derivative” of a reference polypeptide sequence may include a deletion relative to the reference polypeptide sequence.

Regarding proteins, “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide; in other embodiments, a fragment may comprise less than about 5, 10, 15, 20, 25, 30, 40, 50, 60, 70, 80, 90, 100, 150, 250, or 500 contiguous amino acid residues of a reference polypeptide; or in other embodiments, a fragment has a length within a range bounded by any of these values (e.g., a range of 50-100 contiguous amino acids of a reference polypeptide). Fragments may be preferentially selected from certain regions of a entity. The term “at least a fragment” encompasses the full length polypeptide. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues, or a range of amino acid residues bounded by any of these values (e.g., an insertion or addition of 5-10 amino acids). A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

Regarding proteins, as used herein, “chimeric proteins,” “chimeric peptides,” “fusion proteins,” or “fusion peptides” refer to proteins or peptides created through the linking two or more functional domains from separate or same proteins via an amino acid linker, resulting in a single polypeptide with functional properties derived from each of the original proteins. The linker can be 10-50 amino acids in length and is rich in glycine for flexibility, as well as serine or threonine for solubility. A “variant” of a reference polypeptide sequence may include a fusion polypeptide comprising the reference polypeptide.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases. As described herein, variants, mutants, or fragments (e.g., a protein variant, mutant, or fragment thereof) may have 99%, 98%, 97%, 96%, 95%, 94%, 93%, 92%, 91%, 90%, 80%, 70%, 60%, 50%, 40%, 30%, or 20% amino acid sequence identity relative to a reference entity.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference entity. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following table provides a list of exemplary conservative amino acid substitutions which are contemplated herein:

| Original | |
|---|---|
| Residue | Conservative Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asp, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the entity at the site of the substitution, and/or (c) the bulk of the side chain.

The disclosed proteins, mutants, variants, or described herein may have one or more functional or biological activities exhibited by a reference polypeptide (e.g., one or more functional or biological activities exhibited by wild-type protein). For example, the disclosed proteins, mutants, variants, or derivatives thereof may have one or more biological activities that include binding to the small entity ABA and targeting an EV to a target cell.

The disclosed proteins may be substantially isolated or purified. The term “substantially isolated or purified” refers to proteins that are removed from their natural environment, and are at least 60% free, preferably at least 75% free, and more preferably at least 90% free, even more preferably at least 95% free from other components with which they are naturally associated.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, the term “targeting domain” or “targeting peptide” refers to peptide moieties that will facilitate specific binding of the EV to a target cell. Sample “targeting domain” or “targeting peptide” include but are not limited to antibodies and any antibody fragments or antigen binding fragments, e.g., Fab, Fab′ and F(ab′)₂, Fd, single-chain Fvs (scFv), single-chain antibodies, disulfide-linked Fvs (sdFv), a de novo-designed binding molecule, affinibody, a DARPIN, and nanobody. Those antibody fragments are well-known to a skilled person in the art.

### Compositions of Present Technology

In one aspect, the present disclosure provides a chimeric protein or peptide comprising: (a) a cargo entity; and (b) a cargo-loading domain comprising an abscisic acid-insensitive 1 (ABI1) sequence.

In some embodiments, the chimeric protein or peptide further comprises a linker that connects the cargo entity and the cargo-loading domain. In some embodiments, the linker comprises amino acids sequence of (GGGGS)ₙ, wherein n is 1, 2, 3, 4, 5, 6, 7, 8, 9, 10, 11, 12, 13, 14, 15, or more. In some embodiments, the linker comprises: (1) an amino acid sequence selected from SEQ ID NO: 10 (TSGGGGSGGGSGGGS), SEQ ID NO: 12 (TRGGGGSGGGSGGGS), SEQ ID NO: 14 (GGGGSGGGSGGGSTG), SEQ ID NO: 15 (DQSNSEEAKKEEAKKEEAKKSNS), SEQ ID NO: 16 (SGGGSGGGSGGGSGGSGGSGGGSGGSGGSGGGSGGGSGGG), and SEQ ID NO: 17 (ESKYGPPAPPAP); or (2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 10, 12, 14, 15, 16, or 17.

In some embodiments, the cargo-loading domain is a truncated variant of a wild-type protein that comprises an extracellular vesicle targeting domain. In some embodiments, the cargo-loading domain comprises residues 126-423 of wild type ABI1. In some embodiments, the cargo-loading domain comprises: MTRVPLYGFTSICGRRPEMEAAVSTIPRFLQSSSGSMLDGRFDPQSAAHFFGVYDGHGG SQVANYCRERMHLALAEEIAKEKPMLCDGDTWLEKWKKALFNSFLRVDSEIESVAPET VGSTSVVAVVFPSHIFVANCGDSRAVLCRGKTALPLSVDHKPDREDEAARIEAAGGKVI QWNGARVFGVLAMSRSIGDRYLKPSIIPDPEVTAVKRVKEDDCLILASDGVWDVMTDE EACEMARKRILLWHKKNAVAGDASLLADERRKEGKDPAAMSAAEYLSKLAIQRGSKD NISVVVVDLK (SEQ ID NO: 6), VPLYGFTSICGRRPEMEAAVSTIPRFLQ SSSGSMLDGRFDPQSAAHFFGVYDGHGGSQV ANYCRERMHLALAEEIAKEKPMLCDGDTWLEKWKKALFNSFLRVDSEIESVAPETVGS TSVVAVVFPSHIFVANCGDSRAVLCRGKTALPLSVDHKPDREDEAARIEAAGGKVIQWN GARVFGVLAMSRSIGDRYLKPSIIPDPEVTAVKRVKEDDCLILASDGVWDVMTDEEACE MARKRILLWHKKNAVAGDASLLADERRKEGKDPAAMSAAEYLSKLAIQRGSKDNISVV VVDLK (SEQ ID NO: 7), a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 6 or 7, or a functional fragment of SEQ ID NO: 6, SEQ ID NO: 7, or a variant amino acid sequence thereof. For the purposes of the present disclosure, a functional fragment of ABI1 may be about 5 amino acids long, about 10 amino acids long, about 15 amino acids long, about 20 amino acids long, about 25 amino acids long, about 30 amino acids long, about 35 amino acids long, about 40 amino acids long, about 45 amino acids long, about 50 amino acids long, about 55 amino acids long, about 60 amino acids long, about 65 amino acids long, about 70 amino acids long, about 75 amino acids long, about 80 amino acids long, about 85 amino acids long, about 90 amino acids long, about 95 amino acids long, about 100 amino acids long, about 105 amino acids long, about 110 amino acids long, about 115 amino acids long, about 120 amino acids long, about 125 amino acids long, about 130 amino acids long, about 135 amino acids long, about 140 amino acids long, about 145 amino acids long, about 150 amino acids long, about 155 amino acids long, about 160 amino acids long, about 165 amino acids long, about 170 amino acids long, about 175 amino acids long, about 180 amino acids long, about 185 amino acids long, about 190 amino acids long, about 195 amino acids long, about 200 amino acids long, about 205 amino acids long, about 210 amino acids long, about 215 amino acids long, about 220 amino acids long, about 225 amino acids long, about 230 amino acids long, about 235 amino acids long, about 240 amino acids long, about 245 amino acids long, or about 250 amino acids long. In other words, a functional fragment may be 5-50 amino acids, 5-40 amino acids, 5-30 amino acids, 5-20 amino acids, 5-15 amino acids, 10-50 amino acids, 10-40 amino acids, 10-30 amino acids, or 10-20 amino acids. In general, a fragment is considered a functional fragment if it is capable of increasing active loading of the cargo entity to the lipid bilayer particle (e.g., CDMP), binding to an ABI1-binding protein, or a combination thereof.

### Cargo Entity

In some embodiments, disclosed technologies comprise a cargo entity. In some embodiments, a lipid bilayer particle or a population of lipid bilayer particles comprises a cargo entity. In some embodiments, disclosed technologies comprise one or more cargo entities, such as multiple cargo entities (e.g., a first cargo entity, a second cargo entity, etc. or a combination thereof). Cargo entities described herein can of any chemical class, e.g., polypeptides, nucleic acids, saccharides, lipids, small entities, and combinations thereof. In some embodiments, a cargo entity is a cargo molecule. In some embodiments, cargo entities are viral nucleocapsids or derivatives thereof. In some embodiments, a cargo entity comprises a viral nucleocapsid, a synthetic nucleic acid, a transcription factor, a recombinase, a base editor, a prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, or a ribonucleoprotein complex.

In some embodiments, a cargo entity is a macromolecular assembly (e.g., a viral capsid, a VLP, a viral particle, or fragments thereof).

In some embodiment's, a cargo entity is an intraparticular macromolecular assembly (e.g., lentiviral cores, AAV particles, other viral cores, VLP cores, and subunits thereof).

In some embodiments, a cargo entity is a nucleocapsid. In some embodiments, a nucleocapsid is a viral nucleocapsid. In some embodiments, a nucleocapsid is a recombinant viral nucleocapsid. In some embodiments, a nucleocapsid comprises cargo nucleic acids and cargo polypeptides.

In some embodiments, a cargo entity is or encodes an AAV nucleocapsid, or a LVV nucleocapsid, or fragments thereof.

In some embodiments, the cargo entity is a cytosolic cargo entity or a membrane bound cargo entity. For example, in some embodiments the cargo-loading domain comprising an abscisic acid-insensitive 1 (ABI1) sequence may be fused to a membrane protein or peptide (i.e., a membrane-bound protein or peptide). In other words, in some embodiments, the cargo entity may be a membrane protein or peptide. Similarly, in some embodiments the cargo-loading domain comprising an abscisic acid-insensitive 1 (ABI1) sequence may be fused to a cytosloic protein or peptide (i.e., a protein or peptide that is not membrane-bound). In other words, in some embodiments, the cargo entity may be a cytosolic protein or peptide.

In another aspect, the present disclosure provides a loading system for loading a desired cargo (or cargos) into a lipid bilayer particle (e.g., CDMP), such as an extracellular vesicle (e.g., exosome or microsome), virus particles, virus-like particles (VLPs), apoptotic bodies, and platelet-like particles. The loading system may be used to deliver a single cargo (or single type of cargo) to a desired cell, or it may be used to deliver multiple cargos (e.g., different proteins or peptides) to a desired cell. For the purposes of embodiments that include multiple different cargos, the disclosed loading system can be used to control the ratios of the various cargos loaded into the lipid bilayer particle (e.g., CDMP) and, subsequently, delivered to a desired cell.

The disclosed lipid bilayer particle (e.g., CDMP) loading system can comprise the chimeric protein or peptide of any one of the above embodiments, and a second chimeric protein comprising (i) a second cargo entity or a targeting molecule, and (ii) and an abscisic acid (ABA)-binding sequence, wherein the second chimeric protein or peptide optionally comprises a second linker that connects the second cargo entity and the ABA-binding sequence. In some embodiments, the second chimeric protein or peptide is a membrane-bound protein or peptide. In some embodiments, the second chimeric protein or peptide is a cytosolic protein or peptide.

In some embodiments, the ABA-binding sequence of the second chimeric protein or peptide comprises a pyrabactin resistance 1-like (PYL1) sequence. In some embodiments, the PYL1 sequence comprises residues 33-209 of wild type PYL1. In some embodiments, the PYL1 sequence comprises MGGGAPTQDEFTQLSQSIAEFHTYQLGNGRCSSLLAQRIHAPPETVWSVVRRFDRPQIY KHFIKSCNVSEDFEMRVGCTRDVNVISGLPANTSRERLDLLDDDRRVTGFSITGGEHRLR NYKSVTTVHRFEKEEEEERIWTVVLESYVVDVPEGNSEEDTRLFADTVIRLNLQKLASIT EAMN (SEQ ID NO: 2), TQDEFTQLSQSIAEFHTYQLGNGRCSSLLAQRIHAPPETVWSVVRRFDRPQIYKHFIKSCN VSEDFEMRVGCTRDVNVISGLPANTSRERLDLLDDDRRVTGFSITGGEHRLRNYKSVTT VHRFEKEEEEERIWTVVLESYVVDVPEGNSEEDTRLFADTVIRLNLQKLASITEAMN (SEQ ID NO: 3), a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 2 or 3, or a functional fragment of SEQ ID NO: 2, SEQ ID NO: 3, or a variant amino acid sequence thereof. For the purposes of the present disclosure, a functional fragment of a ABA-binding sequence may be about 5 amino acids long, about 10 amino acids long, about 15 amino acids long, about 20 amino acids long, about 25 amino acids long, about 30 amino acids long, about 35 amino acids long, about 40 amino acids long, about 45 amino acids long, about 50 amino acids long, about 55 amino acids long, about 60 amino acids long, about 65 amino acids long, about 70 amino acids long, about 75 amino acids long, about 80 amino acids long, about 85 amino acids long, about 90 amino acids long, about 95 amino acids long, about 100 amino acids long, about 105 amino acids long, about 110 amino acids long, about 115 amino acids long, about 120 amino acids long, about 125 amino acids long, about 130 amino acids long, about 135 amino acids long, about 140 amino acids long, about 145 amino acids long, about 150 amino acids long, about 155 amino acids long, about 160 amino acids long, about 165 amino acids long, about 170 amino acids long, about 175 amino acids long, about 180 amino acids long, about 185 amino acids long, about 190 amino acids long, about 195 amino acids long, about 200 amino acids long, about 205 amino acids long, about 210 amino acids long, about 215 amino acids long, about 220 amino acids long, about 225 amino acids long, about 230 amino acids long, about 235 amino acids long, about 240 amino acids long, about 245 amino acids long, or about 250 amino acids long. In other words, a functional fragment may be 5-50 amino acids, 5-40 amino acids, 5-30 amino acids, 5-20 amino acids, 5-15 amino acids, 10-50 amino acids, 10-40 amino acids, 10-30 amino acids, or 10-20 amino acids. In general, a fragment is considered a functional fragment if it is capable of increasing active loading of the cargo entity to the lipid bilayer particle (e.g., CDMP), binding to ABI1 or a variant or fragment thereof, or a combination thereof.

In some embodiments, the second linker comprises amino acids sequence of (GGGGS), wherein n is 1, 2, 3, 4, 5, 6, 7, 8, 9, 10, 11, 12, 13, 14, 15, or more. In some embodiments, the second linker comprises: (1) an amino acid sequence selected from SEQ ID NO: 10, SEQ ID NO: 12, SEQ ID NO: 14, SEQ ID NO: 15, SEQ ID NO: 16, and SEQ ID NO: 17; or (2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 10, 12, 14, 15, 16, or 17.

In some embodiments, the lipid bilayer particle (e.g., CDMP) loading system of any one of the above embodiments further comprises abscisic acid (ABA), wherein the cargo-loading domain of the chimeric protein or peptide and/or the ABA-binding sequence of the second chimeric protein or peptide can bind to ABA, leading to dimerization of the chimeric protein or peptide and the second chimeric protein or peptide.

In some embodiments, the second cargo entity is a cytosolic cargo entity or a membrane bound cargo entity.

In some embodiments, a cargo entity is a polypeptide cargo entity. In some embodiments, the first or the second cargo entity is a cytosolic cargo molecule, wherein the cytosolic cargo entity may be any peptide, polypeptide, or protein of interest to be delivered to a target cell, such as an enzyme, a therapeutic agent (e.g., an antibody, inhibitor, an agonist, and an antagonist), or a fluorescent protein. In some embodiments, the cytosolic cargo entity can be selected from any one or more of base editors, prime editors, TALENs, ZFNs, kinases, kinase inhibitors, activators or inhibitors of receptor-signaling, intrabodies, chromatin-modifying synthetic transcription factors, natural transcription factors, and mutant forms thereof. In some embodiments, the cytosolic cargo entity is a CRISPR enzyme, e.g., a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is a Cas9 protein (e.g., a naturally-occurring bacterial Cas9 as well as any chimeras, mutants, homologs or orthologs). Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof.

In some embodiments, a cargo entity is a nucleic acid cargo entity. In some embodiments, the first or the second cargo entity may be a nucleic acid (e.g., DNA or RNA) or another entity. Nucleic acid cargo entities can include, but are not limited to, DNA encoding a protein or peptide of interest, mRNA, siRNA, shRNA, miRNA, an antisense oligonucleotide, and combinations thereof. Other potential cargo entities include, but are not limited to, viral and nonviral vectors that are expressed inside a cell (or can be delivered to the cytosol of a cell), and ribonucleoprotein complexes, such as CRISPR-type entities and endogenous complexes such as DICER or RISC bound to natural or synthetic RNA such as miRNA, shRNA, etc.). Indeed, those skilled in the art will recognize that any cargo that can be expressed in a cell or physically delivered to the inside of a cell can be fused (genetically or synthetically) to an ABI protein/peptide or an ABA-binding sequence and is expressly contemplated here. Genetic fusion can be achieved by expressing in a lipid bilayer particle (e.g., CDMP such as EV) producing cell the two or more components of the chimeric protein or peptide or peptide. Alternatively, the cargo entity fused to a loading domain may be delivered to a lipid bilayer particle (e.g., CDMP such as EV) producing cells leading to subsequent incorporation into the lipid bilayer particles (e.g., CDMPs). Alternatively, the cargo entity fused to a loading domain may be inserted into lipid bilayer particles (e.g., CDMPs) after secretion from producer cells.

Additionally or alternatively, in any of the above embodiments, the first or the second cargo entity may be a membrane bound cargo entity. In some embodiments, the membrane bound cargo entity may comprise (i) a targeting peptide/protein and (ii) a transmembrane domain. Exemplary targeting peptides include but are not limited to any antibody fragments or antigen binding fragments, e.g., Fab, Fab′ and F(ab′)₂, Fd, scFv, single-chain antibodies, disulfide-linked Fvs (sdFv), and nanobodies. The targeting peptide (e.g., scFv) may bind to a target of interest on a specific cell type, such as a T cell. In some embodiments, the targeting peptide may be a Fab, Fab′ and F(ab′)₂, Fd, scFv, single-chain antibodies, disulfide-linked Fvs (sdFv), a de novo-designed binding molecule, affinibody, a DARPIN, or nanobody.

Transmembrane domains are known in the art. Transmembrane domains (TMDs) consist predominantly of nonpolar amino acid residues and may traverse the bilayer once (single pass) or several times. TMDs usually consist of a helices. The peptide bond is polar and can include internal hydrogen bonds formed between carbonyl oxygen atoms and amide nitrogen atoms which may be hydrated. Within the lipid bilayer, where water is essentially excluded, peptides usually adopt the α-helical configuration in order to maximize their internal hydrogen bonding. A length of helix of 18-21 amino acid residues is usually sufficient to span the usual width of a lipid bilayer. TMDs that are oriented with an extracytoplasmic N-terminus and a cytoplasmic C-terminus are classified as type I TMDs, and TMDs that are oriented with an extracytoplasmic C-terminus and a cytoplasmic N-terminus are classified as type II TMDs. In some embodiments of the disclosed extracytoplasmic, they are classified as type I or, if cytoplasmic, type II. In some embodiments, a transmembrane domain is a single pass, type I transmembrane domain comprising 18-21 amino acids, where at least about 90% of the amino acids are nonpolar. Suitable TMDs for the disclosed fusion proteins may include the transmembrane domain of cellular receptors, such as the platelet-derived growth factor receptor (PDGFR) transmembrane domain of SEQ ID NO: 18 (AVGQDTQEVIVVPHSLPFKVVVISAILALVVLTIISLIILIMLWQKKPR), which can be encoded by the nucleotide sequence GCCGTCGGCCAGGACACCCAAGAAGTGATCGTCGTCCCTCACAGCCTGCCTTTCAAG GTGGTGGTCATCAGCGCCATTCTGGCCCTGGTGGTGCTGACCATCATCAGCCTGATC ATCCTGATTATGCTGTGGCAGAAGAAGCCCAGA (SEQ ID NO: 19). The TMD may be linked directly to the targeting peptide (e.g., a scFv) or the TMD may be linked via a linker. In some embodiments, the linker linking the TMD and the targeting peptide/protein comprises amino acids sequence of (GGGGS)ₙ, wherein n is 1, 2, 3, 4, 5, 6, 7, 8, 9, 10, 11, 12, 13, 14, 15, or more. In some embodiments, the linker linking the TMD and the targeting peptide comprises: (1) an amino acid sequence selected from SEQ ID NO: 10, SEQ ID NO: 12, SEQ ID NO: 14, SEQ ID NO: 15, SEQ ID NO: 16, and SEQ ID NO: 17; or (2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 10, 12, 14, 15, 16, or 17.

### Engineered Lipid Bilayer Particles

Among other things, the present disclosure provides engineered lipid bilayer particles, and preparations thereof.

In some embodiments, a lipid bilayer particle (e.g., a CDMP) is an extracellular vesicle, which can be selected from an exosome or a microvesicle. In some embodiments, a lipid bilayer particle (e.g., a CDMP) can be a virus particle, a virus-like particles (VLP), an apoptotic body, and a platelet-like particle. In some embodiments, a lipid bilayer particle (e.g., CDMP) can be a hybrid particle generated by mixing a cell-derived particle or vesicle (e.g., a particle or vesicle that blebbed or budded from a cell) and a synthetic vesicle.

In another aspect, the present disclosure provides an extracellular vesicle comprising the chimeric peptide or the extracellular vesicle targeting system of any one of the above embodiments. In some embodiments, extracellular vesicles are selected from exosomes, microvesicles and combinations thereof.

In another aspect, the present disclosure provides a nucleic acid encoding the chimeric peptide or the lipid bilayer particle targeting system of any one of the above embodiments.

In some embodiments, a lipid bilayer particle targeting peptide of the first chimeric peptide is encoded by ATGACCAGAGTGCCCCTGTACGGCTTCACCAGCATTTGTGGCAGACGGCCCGAAAT GGAAGCCGCCGTGTCTACAATCCCCAGATTCCTCCAGAGCAGCAGCGGCTCCATGCT GGACGGCAGATTCGATCCTCAGAGCGCCGCTCACTTCTTCGGCGTGTACGATGGACA TGGCGGAAGCCAGGTGGCCAACTACTGCCGCGAAAGAATGCATCTGGCCCTGGCCG AGGAAATCGCCAAAGAAAAGCCCATGCTGTGCGACGGCGACACCTGGCTGGAAAA GTGGAAGAAGGCCCTGTTCAACAGCTTCCTGAGAGTGGACAGCGAGATCGAGAGCG TGGCCCCTGAAACAGTGGGCAGCACATCTGTGGTGGCCGTGGTGTTTCCCAGCCACA TCTTCGTGGCTAACTGCGGCGATAGCAGAGCCGTGCTGTGCAGAGGAAAAACAGCC CTGCCTCTGTCCGTGGACCACAAGCCTGATAGAGAGGATGAGGCCGCCAGAATTGA AGCCGCTGGCGGCAAAGTGATCCAGTGGAATGGCGCTAGAGTGTTCGGCGTGCTGG CCATGAGTAGATCCATCGGCGATAGATACCTGAAGCCTAGCATCATCCCCGATCCTG AAGTGACCGCCGTGAAGAGAGTGAAAGAGGACGACTGCCTGATCCTGGCCTCTGAC GGTGTCTGGGACGTGATGACAGATGAAGAGGCCTGCGAGATGGCCCGGAAGAGAAT CCTGCTGTGGCACAAGAAAAACGCCGTGGCCGGGGATGCTTCTCTGCTGGCTGACG AGAGAAGAAAAGAGGGCAAAGACCCCGCTGCCATGTCTGCCGCCGAGTACCTGTCT AAGCTGGCCATCCAGAGAGGCAGCAAGGACAACATCAGCGTGGTGGTCGTGGACCT GAAA (SEQ ID NO: 5), a variant nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 5, or a functional fragment thereof. In general, a fragment is considered a functional fragment if it encodes a protein or peptide that is capable of increasing active loading of the cargo entity to the lipid bilayer particle (e.g., CDMP), binding to an ABI1-binding protein, or a combination thereof.

In some embodiments, the ABA-binding sequence of the second chimeric peptide is encoded by ATGGGCGGAGGAGCCCCTACCCAGGACGAGTTCACCCAGCTGAGCCAGAGCATCGC TGAGTTCCACACCTACCAGCTGGGAAACGGACGCTGTTCCAGCCTGCTGGCACAGA GAATCCACGCTCCTCCTGAGACAGTGTGGAGTGTGGTGCGCAGATTCGACCGCCCTC AGATTTACAAGCACTTCATCAAGAGCTGCAACGTGAGCGAGGACTTCGAGATGAGA GTGGGATGTACCAGAGATGTGAACGTGATCAGCGGACTGCCTGCCAACACCAGCAG AGAGAGACTGGACCTGCTGGACGATGACCGCAGAGTGACCGGCTTCAGCATCACCG GAGGTGAGCACAGACTGAGAAACTACAAGAGCGTGACCACCGTCCACCGCTTCGAG AAGGAAGAGGAAGAGGAGCGCATCTGGACCGTGGTGCTGGAGAGCTACGTCGTGG ACGTGCCCGAGGGCAACAGCGAAGAGGATACCCGCCTGTTCGCTGACACCGTGATC AGACTGAACCTCCAGAAGCTGGCCAGCATCACCGAGGCAATGAAC (SEQ ID NO: 1), a variant nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, or a functional fragment thereof. In general, a fragment is considered a functional fragment if it encodes a protein or peptide that is capable of increasing active loading of the cargo entity to the lipid bilayer particle (e.g., CDMP), binding to ABI1 or a variant or fragment thereof, or a combination thereof.

In some embodiments, the linker and/or second linker are encoded by one of SEQ ID NOs: 9 (ACTAGTGGCGGCGGAGGCAGCGGAGGCGGATCTGGCGGAGGATCT), 11 (ACGCGTGGCGGCGGAGGCAGCGGAGGCGGATCTGGCGGAGGATCT), or 13 (GGCGGCGGAGGAAGTGGCGGCGGATCTGGCGGAGGATCTACCGGT), or a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to one of SEQ ID NOs: 9, 11, or 13.

In another aspect, the present disclosure provides a cell comprising the chimeric protein or peptide, the lipid bilayer particle (e.g., CDMP) loading system, the lipid bilayer particle (e.g., CDMP such as extracellular vesicle), or the nucleic acid of any one of the above embodiments. In some embodiments, the cell is a mammalian cell. Suitable mammalian cells include, but are not limited to, HEK293, HEK293FT, PER.C6, mesenchymal stem cells, megakaryocytes, iPSCs, T cells, erythrocytes and erythropoetic precursors, and iPSC-derived version of any of the preceding cells.

### Methods of Making and Using

In another aspect, the present disclosure provides a method of loading a cargo entity into a lipid bilayer particle (e.g., CDMP), such as an extracellular vesicle (EV), comprising expressing in a cell the chimeric peptide of any one of the above embodiments. For example, the method of loading a cargo entity into the lipid bilayer particle (e.g., CDMP such as EV) may comprise expressing in a eukaryotic cell (a) an mRNA that encodes the chimeric peptide comprising the cargo entity and (b) expressing in the eukaryotic cell the chimeric peptide. The mRNA for the chimeric peptide comprising the cargo entity may be expressed from vectors that are transfected into suitable production cells for producing the disclosed extracellular vesicles. Note that the vector may also be stably transfected. The vector or vectors for expressing the mRNA for the chimeric peptide comprising the cargo entity may be packaged in a kit designed for preparing the disclosed extracellular vesicles.

In some embodiments, loading of the cargo entity of the chimeric peptide is enhanced compared to passive cargo loading. For example, the claimed methods can achieve up to a 23-fold enrichment of cargo entities in microvesicles and up to a 49-fold enrichment in exosomes compared to passive loading in these respective particles.

In some embodiments, the cargo entity can be any polypeptide of interest to be delivered to a target cell, such as an enzyme, a therapeutic agent (e.g., an antibody, inhibitor, an agonist, and an antagonist), or a fluorescent protein. In some embodiments, the cargo entity is a base editor, a prime editor, TALEN, ZFN, kinase, kinase inhibitor, activator or inhibitor of receptor-signaling, intrabody, chromatin-modifying synthetic transcription factor, natural transcription factor, and mutant forms thereof. In some embodiments, the cargo entity is a CRISPR enzyme, e.g., a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is any Cas9 protein, for instance any naturally-occurring bacterial Cas9 as well as any chimeras, mutants, homologs or orthologs. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof.

In some embodiments, the cargo entity may be a nucleic acid (e.g., DNA or RNA) or another entity. Nucleic acid cargo entities can include, but are not limited to, DNA encoding a protein or peptide of interest, mRNA, siRNA, shRNA, miRNA, an antisense oligonucleotide, and combinations thereof. Other potential cargo entities include, but are not limited to, viral and nonviral vectors that are expressed inside a cell (or can be delivered to the cytosol of a cell), and ribonucleoprotein complexes, such as CRISPR-type entities and endogenous complexes such as DICER or RISC bound to natural or synthetic RNA such as miRNA, shRNA, etc.). Indeed, those skilled in the art will recognize that any cargo that can be expressed in a cell or physically delivered to the inside of a cell can be fused (genetically or synthetically) to an ABI protein/peptide or an ABA-binding sequence and is expressly contemplated here.

In another aspect, the present disclosure provides a method of loading two cargo molecules into a lipid bilayer particle (e.g., CDMP), such as an extracellular vesicle (EV), comprising expressing in a cell the lipid bilayer particle (e.g., CDMP such as EV) a loading system of any one of the above embodiments.

In some embodiments, co-localization of the cargo entity of the chimeric protein or peptide or peptide and the second cargo entity of the second chimeric protein or peptide or peptide is enhanced compared to passive cargo loading.

In some embodiments, the cargo entity can be any polypeptide of interest to be delivered to a target cell, such as an enzyme, a therapeutic agent (e.g., an antibody, inhibitor, an agonist, and an antagonist), or a fluorescent protein. In some embodiments, the cargo entity is a base editor, a prime editor, TALEN, ZFN, kinase, kinase inhibitor, activator or inhibitor of receptor-signaling, intrabody, chromatin-modifying synthetic transcription factor, natural transcription factor, and mutant forms thereof. In some embodiments, the cargo entity is a CRISPR enzyme, e.g., a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is any Cas9 protein, for instance any naturally-occurring bacterial Cas9 as well as any chimeras, mutants, homologs or orthologs. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof.

In some embodiments, the cargo entity may be a nucleic acid (e.g., DNA or RNA) or another entity. Nucleic acid cargo entities can include, but are not limited to, DNA encoding a protein or peptide of interest, mRNA, siRNA, shRNA, miRNA, an antisense oligonucleotide, and combinations thereof. Other potential cargo entities include, but are not limited to, viral and non-viral vectors that are expressed inside a cell (or can be delivered to the cytosol of a cell), and ribonucleoprotein complexes, such as CRISPR-type entities and endogenous complexes such as DICER or RISC bound to natural or synthetic RNA such as miRNA, shRNA, etc.). Indeed, those skilled in the art will recognize that any cargo that can be expressed in a cell or physically delivered to the inside of a cell can be fused (genetically or synthetically) to an ABI protein/peptide or an ABA-binding sequence and is expressly contemplated here.

Also contemplated herein are methods for using the disclosed extracellular vesicles. For example, the disclosed extracellular vesicles may be used for delivering one or more cargo proteins a target cell, where the methods include contacting the target cell with the disclosed extracellular vesicles. The disclosed extracellular vesicles may be formulated as part of a pharmaceutical composition for treating a disease or disorder and the pharmaceutical composition may be administered to a patient in need thereof to deliver the cargo entities to target cells in order to treat the disease or disorder.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Methods and Materials

Plasmid construction. Plasmids were constructed using standard molecular biology techniques. Codon optimization was performed using the GeneArt gene synthesis tool (Thermo Fisher). PCR was performed using Phusion DNA polymerase (New England Biolabs, NEB), and plasmid assembly was performed via restriction enzyme cloning. Plasmids were transformed into TOP10 competent E. Coli (Thermo Fisher) and grown at 37° C.

Plasmid backbones. A modified pcDNA3.1 (Thermo Fisher V87020), was used to generate a general expression vector. Briefly, the hygromycin resistance gene and SV40 promoter were removed, leaving the SV40 origin of replication and poly(A) signal intact. The BsaI sites in the AmpR gene and 5′-UTR and the BpiI site in the bGH poly(A) signal were mutated. The lentiviral vector pGIPZ (Open Biosystems) was obtained through the Northwestern High Throughput Analysis Laboratory. PlentiCRISPRv2 was a gift from Feng Zhang⁷⁴ (Addgene plasmid No. 52961).

Plasmid source vectors. Fluorescent proteins enhanced blue fluorescent protein 2 (EBFP2), enhanced yellow fluorescent protein (EYFP), and dimeric tomato (dTomato) were sourced from Addgene vectors (plasmid Nos. 14893, 58855, and 18917, respectively) gifted by Robert Campbell,⁷⁵ Joshua Leonard,⁷⁶ and Scott Sternson.⁷⁷ Monomeric teal fluorescent protein 1 (TFP1) was synthesized by Thermo Fisher. The scFv was synthesized from a previously published scFv sequence and the PDGFR transmembrane domain was sourced from a pDisplay system vector (Addgene plasmid No. 61556, gifted by Robert Campbell).⁷⁸ The C1C2 domain sequence was provided by Natalie Tigue³⁵ and synthesized by Thermo Fisher. Constitutively active Cx43 and SLAM were synthesized by Thermo Fisher from Uniprot sequences P17302 CXA1_HUMAN and Q13291-1 SLAF1_THUMAN isoform 1, respectively. pX330 encoding Cas9 was gifted by Erik Sontheimer (UMass), originally sourced from Addgene plasmid No. 42230 gifted by Feng Zhang.⁷⁹ The CXCR4 sgRNA sequence was provided by Judd Hultquist and is as follows: GAAGCGTGATGACAAAGAGG.⁶³ ABI and PYL domains⁵⁰ were synthesized by Thermo Fisher and IDT, respectively.

Plasmid preparation. Bacteria were grown overnight in 100 mL LB+Amp cultures for 12-14 h. Cultures were spun at 3,000 g for 10 min to pellet the bacteria, and pellets were resuspended and incubated for 30 min in 4 mL of 25 mM Tris pH 8.0, 10 mM EDTA, 15% sucrose, and 5 mg/mL lysozyme. Bacteria were lysed for 15 min in 8 mL of 0.2 M NaOH and 1% SDS, followed by a 15 min neutralization in 5 mL of 3 M sodium acetate (pH 5.2). The precipitate was pelleted at 9,000 g for 20 min, and supernatant was filtered through cheese cloth and incubated for 1-3 h at 37° C. with 3 μL of 10 mg/mL RNAse A (Thermo Fisher). Samples were extracted with 5 mL phenol chloroform, and the aqueous layer was recovered after centrifugation at 7,500 g for 20 min. A second phenol chloroform extraction was performed with 7 mL solvent. 0.7 volumes isopropanol was added to the recovered supernatant, and samples were inverted and incubated at room temperature for 10 min prior to centrifugation at 9,000 g for 20 min to pellet the DNA mixture. Pellets were briefly dried and resuspended in 1 mL of 6.5% PEG 20,000 and 0.4 M NaCl. DNA was incubated on ice overnight and pelleted at 21,000 g for 20 min. The supernatant was removed, and pellets were washed in cold absolute ethanol and dried at 37° C. before resuspension in TE buffer (10 mM Tris, 1 mM EDTA, pH 8.0). DNA was diluted to 1 μg/μL using a Nanodrop 2000 (Thermo Fisher).

Cell culture. HEK293FT cells (Thermo Fisher R70007) were cultured in Dulbecco's Modified Eagle Medium (DMEM, Gibco 31600-091) supplemented with 10% FBS (Gibco 16140-071), 1% penicillin-streptomycin (Gibco 15140-122), and 4 mM additional L-glutamine (Gibco 25030-081). Jurkat T cells (ATCC TIB-152) were cultured in Roswell Park Memorial Institute Medium (RPMI 1640, Gibco 31800-105) supplemented with 10% FBS, 1% pen-strep, and 4 mM L-glutamine. Sublines generated from these cell lines were cultured in the same way. Cells were subcultured at a 1:5 or 1:10 ratio every 2-3 d, using Trypsin-EDTA (Gibco 25300-054) to remove adherent cells from the plate. Lenti-X cells (Takara) were cultured the same way with additional 1 mM sodium pyruvate (Thermo Fisher 11360-070). Primary human CD4+T cells were cultured in RPMI supplemented with 10% FBS, 1% pen-strep, 5 mM HEPES, 5 mM sodium pyruvate, and 20U/mL IL-2 (added fresh at time of use). Cells were maintained at 37° C. at 5% CO₂. HEK293FT and Jurkat cells tested negative for mycoplasma with the MycoAlert Mycoplasma Detection Kit (Lonza LT07-318).

Transfection. For transfection of HEK293FT cells and derived cell lines in 15 cm dishes for EV packaging, cells were plated at a density of 18×10⁶ cells/dish (1×10⁶ cells/mL) 6-12 h prior to transfection. Cells were transfected with 30 μg DNA plus 1 μg of a fluorescent transfection control via the calcium phosphate method. Plasmid DNA was mixed with 2 M CaCl₂) (final concentration 0.3 M) and added to a 2×HEPES-buffered saline solution (280 mM NaCl, 0.5 M HEPES, 1.5 mM Na₂HPO₄) dropwise in a 1:1 ratio and mixed seven times by pipetting. The transfection solution was incubated for 3 min, mixed eight times by pipetting, and added gently to the side of the plate. For transfection of HEK293FT cells in 10 cm dishes for EV packaging, cells were plated at a density of 5×10⁶ cells/dish (6.25×10⁵ cells/mL) and transfected with 20 μg DNA plus 1 μg transfection control as described above, adding transfection mixture dropwise to the dish. Lenti-X cells were transfected in 10 cm dishes in the same manner, though were plated 24 h prior to transfection as per the manufacturer recommendation (Takara). For transfection of HEK293FT cells in 24 well plates, cells were plated at a density of 1.7×10⁵ cells/well (3.4×10⁵ cells/mL) and transfected with 200 μg DNA as described above, adding transfection mixture dropwise to the well. Medium was changed 12-16 h later. Jurkat lipofectamine transfections were performed according to the manufacturer's protocol.

Cell line generation. To generate lentivirus, HEK293FT or Lenti-X cells were plated in 10 cm dishes at a density of 5×10⁶ cells/dish (6.25×10⁵ cells/mL). 6-12 h later for HEK293FT or 24 h later for Lenti-X, cells were transfected with 10 μg of viral vector, 8 μg psPAX2, and 3 μg pMD2G via calcium phosphate transfection as described above. Medium was changed 12-16 h later. 28 h post media change, lentivirus was harvested from the conditioned medium. Medium was centrifuged at 500 g for 2 min to clear cells, and the supernatant was filtered through a 0.45 μm pore filter (VWR). Lentivirus was concentrated from the filtered supernatant by ultracentrifugation in Ultra Clear tubes (Beckman Coulter 344059) at 100,420 g at 4° C. in a Beckman Coulter Optima L-80 XP ultracentrifuge using an SW41Ti rotor. Supernatant was aspirated, leaving virus in ˜100 μL final volume, and concentrated lentivirus was left on ice for at least 30 min prior to resuspension, then used to transduce ˜1×10⁵ cells, either plated at the time of transduction or the day before. When appropriate, drug selection began 2 d post transduction, using antibiotic concentrations of 1 μg/mL puromycin (Invitrogen ant-pr) and 10 μg/mL blasticidin (Alfa Aesar J61883) on HEK293FT cells or 0.2 μg/mL puromycin and 2 μg/mL blasticidin on Jurkat cells. Cells were kept in antibiotics for at least two weeks with subculturing every one to two days.

Sorting of Cas9 reporter lines. Cells were prepared for fluorescence-activated cell sorting (FACS) by resuspending in either DMEM or RPMI, as appropriate, supplemented with 10% FBS, 25 mM HEPES, and 100 μg/mL gentamycin (Amresco 0304) at a concentration of 1×10⁷ cells/mL. Cells were sorted for the highest mTFP1 expressors (top 10% or less) lacking any dTomato expression on a BD FacsAria Ilu using a 488 nm laser (530/30 filter) and a 562 nm laser (582/15 filter). Cells were collected in DMEM or RPMI, as appropriate, supplemented with 20% FBS, 25 mM HEPES, and 100 μg/mL gentamycin. Cells were spun down and resuspended in normal growth medium with 100 μg/mL gentamycin for recovery.

EV production, isolation, and characterization. EV producer cell lines were plated in 10 or 15 cm dishes and transfected the same day by the calcium phosphate method where appropriate. Medium was changed to EV-depleted medium the following morning. EV-depleted medium was made by supplementing DMEM with 10% exosome depleted FBS (Gibco A27208-01), 1% pen-strep, and 4 mM L-glutamine. EVs were harvested from the conditioned medium 24-36 h post medium change by differential centrifugation as previously described.^{41,42} Briefly, conditioned medium was cleared of debris by centrifugation at 300 g for 10 min to remove cells followed by centrifugation at 2,000 g for 20 min to remove dead cells and apoptotic bodies. Supernatant was centrifuged at 15,000 g for 30 min in a Beckman Coulter Avanti J-26XP centrifuge with a J-LITE JLA 16.25 rotor to pellet microvesicles. Supernatant was collected and exosomes pelleted by ultracentrifugation at 120,416 g for 135 min in a Beckman Coulter Optima L-80 XP ultracentrifuge with an SW41 Ti rotor, using polypropylene ultracentrifuge tubes (Beckman Coulter 331372). All centrifugation steps were performed at 4° C. EV pellets were left in ˜100-200 μL of conditioned media and incubated on ice for at least 30 min after supernatant removal before resuspension. EV concentration was determined by NanoSight analysis. Samples were diluted in PBS to concentrations on the order of 10⁸ particles/mL for analysis. NanoSight analysis was performed on an NS300 (Malvern), software version 3.4. Three 30 s videos were acquired per sample using a 642 nm laser on a camera level of 14, an infusion rate of 30, and a detection threshold of 7. Default settings were used for the blur, minimum track length, and minimum expected particle size. EV concentrations were defined as the mean of the concentrations calculated from each video. Size distributions were generated by the software. For TEM, samples were fixed for 10 min in Eppendorf tubes by adding 65 μL of 4% PFA to 200 μL of EVs. 15 μL of fixed suspension was pipetted onto a plasma cleaned (PELCO easiGlow), formvar/carbon coated grid (EMS 300 mesh). After 10 min, the solution was removed by wicking with a wedge of filter paper, then washed by inverting the grid onto a drop of buffer for 30 seconds twice, followed with diH₂O once. A 2% uranyl acetate (Ted Pella) stain was applied twice and wicked after 30 s. Grids were allowed dry before storing in a grid box until use. Grids were imaged in a JEOL JEM 1230 TEM (JEOL USA) with a 100 KV accelerating voltage. Data was acquired with a Orius SC1000 CCD camera (Gatan). EVs were stored on ice and used within 10 days or stored at −80° C. for long term preservation.

Immunoblotting. For western blot analysis, cells were lysed in RIPA buffer (150 mM NaCl, 50 mM Tris-HCl pH 8.0, 1% Triton X-100, 05% sodium deoxycholate, 0.1% SDS, and one protease inhibitor cocktail tablet (Pierce PIA32953) per 10 mL) and incubated on ice for 30 min. Lysates were cleared by centrifugation at 12,000 g for 20 min at 4° C., and supernatant was harvested. Protein concentration was determined by BCA assay (Pierce) according to the manufacturer's instructions. Samples were normalized by protein content ranging from 1 to 2 μg (for cell lysates) or by vesicle count ranging from 1×10⁷ to 6×10⁸ (for EVs). Samples were heated in Laemmli buffer (60 mM Tris-HCl pH 6.8, 10% glycerol, 2% SDS, 100 mM dithiothreitol, 0.01% bromophenol blue) at 70° C. (for membrane-bound scFv and calnexin) or 95° C. (for Cas9, CD9, CD81, and Alix) for 10 min. Samples were loaded onto 4-15% polyacrylamide gradient Mini-PROTEAN TGX precast protein gels (Bio-Rad) and run at 50 V for 10 min followed by 100 V for 1 h. Protein was transferred to a PVDF membrane (Bio-Rad) at 100 V for 45 min. For anti-FLAG blots, membranes were blocked in 3% milk in TBS (50 mM Tris, 138 mM NaCl, 2.7 mM KCl, pH 8.0) for 30 min. Membranes were washed once in TBS for 5 min, then incubated in primary anti-FLAG antibody (Sigma F1804) diluted 1:1000 in 3% milk in TBS overnight at 4° C. Membranes were washed once for 5 min in TBS and twice in TBST 1 (50 mM Tris, 138 mM NaCl, 2.7 mM KCl, 0.05% Tween 20, pH 8.0) for 5 min each prior to secondary antibody staining. For all other blots, membranes were blocked in 5% milk in TBST 2 (50 mM Tris, 150 mM NaCl, 0.1% Tween 20, pH 7.6) for 1 h. Membranes were incubated in primary antibody diluted in 5% milk in TBST 2 overnight at 4° C. Primary antibodies include anti-HA (Cell Signaling Technology 377245 C29F4, 1:1000), anti-CD9 (Santa Cruz Biotechnology sc-13118, 1:500), anti-CD81 (Santa Cruz Biotechnology sc-23962, 1:500, run in non-reducing conditions), anti-Alix (Abcam Ab117600, 1:500), and anti-calnexin (Abcam Ab22595, 1:1000). Membranes were washed three times in TBST 2 for 5 min each prior to secondary antibody staining. HRP-conjugated anti-mouse (Cell Signaling Technology 7076) and anti-rabbit (Invitrogen 32460) secondary antibodies were diluted 1:3000 in 5% milk in TBST 2. Membranes were incubated in secondary antibody at room temperature for 1 h, then washed three times in TBST 2 (5 min washes). Membranes were probed with Clarity Western ECL Substrate (Bio-Rad) and either exposed to film, which was developed and scanned, or imaged using an Azure c280 imager. Images were cropped using Adobe Illustrator. No other image processing was employed.

Surface immunoblotting. Cells were transferred to FACS tubes (adherent cells were harvested using FACS buffer (PBS pH 7.4 with 0.05% BSA and 2 mM EDTA) prior to staining) with 1 mL of FACS buffer and centrifuged at 150 g for 5 min. Supernatant was decanted, and cells were resuspended in 50 μL of FACS buffer. 10 μL of human IgG (Thermo Fisher 027102) was added, cells were flicked to mix, and were incubated at 4° C. for 5 min. Conjugated primary antibody was then added at the manufacturer's recommended dilution, cells were flicked to mix and incubated at 4° C. for 30 min. Cells were then washed three times with 1 mL of FACS buffer, centrifuging at 150 g for 5 min and decanting the supernatant after each wash. Cells were resuspended in two drops of FACS buffer prior to flow cytometry. For Miltenyi Biotec antibodies, cells were stained at 4° C. for 15 min without blocking and were washed once prior to flow cytometry, as per manufacturer protocol.

EV binding and uptake experiments. Jurkat T cells or primary human CD4+ T cells were incubated with EVs at an EV to cell ratio of 100,000:1 (typically 1×10¹⁰ EVs per 1×10⁵ cells) unless otherwise indicated. For Jurkats, cells were plated in a 48 well plate with 300 μL total volume. For primary T cells, cells were plated in a 96 well plate with 200 μL total volume. Cells were plated at the time of EV addition, and wells were brought to the appropriate volume with RPMI. For binding experiments, cells were incubated for 2 h at 37° C. unless otherwise indicated, then washed three times in FACS buffer, centrifuging at 150 g for 5 min for Jurkat cells or 400 g for 3 min for primary T cells. Cells were resuspended in one drop of FACS buffer prior to flow cytometry. To adsorb EVs to aldehyde/sulfate latex beads (Thermo Fisher), EVs were mixed with beads at a ratio of 1×10⁹ EVs per 2 μL beads diluted 1:10 in PBS. Volumes were normalized across samples with PBS, and beads and EVs were incubated for 15 min at room temperature. Samples were then brought to 200 μL with PBS and allowed to incubate for 2 h at room temperature while rocking. Cells were blocked for 1 h at 37° C. prior or EV incubation where indicated. To prepare for analysis, cells were washed twice in PBS, incubated with two drops of trypsin-EDTA for 5 min at 37° C. to remove surface bound vesicles. Cells were washed with RPMI to quench the trypsin, then washed twice more with FACS buffer prior to analysis.

Confocal microscopy. Cells were transfected via the calcium phosphate method on poly L-lysine coated glass coverslips and mounted on glass slides for imaging. Microscopy images were taken on Leica SP5 II laser scanning confocal microscope using a 100× oil-immersion objective. Bright-field images were acquired at a PMT setting of 443.0 V. A 514 nm laser at 20% intensity and 94% smart gain was used for fluorescence excitation. Emission spectra were captured from 520-540 nm using an HyD sensor. Images were captured at 512×512 resolution at scanning speed of 400 Hz. Pseudocolored fluorescence images were contrast-adjusted in ImageJ such that 4% of pixels were saturated.

Affinity chromatography. Affinity chromatography isolation was performed as previously reported.⁵¹ Briefly, an anti-FLAG affinity column was prepared by loading anti-FLAG M2 affinity gel (Sigma A2220-1 ML) in a 4 mL 1×5 cm glass column (Bio-Rad) and drained via gravity flow. The column was washed with 5 mL TBS (50 mM Tris-HCl, 150 mM NaCl, pH 7.5) and equilibrated with three sequential 1 mL washes with regeneration buffer (0.1 M glycine-HCl, pH 3.5), followed by a 5 mL wash of TBS. Concentrated EVs were loaded onto the top of the column and chased with 1-2 mL of TBS. The column was incubated with EVs for 5 min before continuing. The flow through was then re-loaded onto the column such that the EV-containing medium passed through the matrix five times. The column was washed with 10 mL TBS prior to elution. EVs were eluted with 2.5 mL elution buffer (100 μg/mL 3×FLAG peptide (Sigma F4799-4 MG) in TBS), which was incubated on the column for 5-10 min after the void fraction was drained (˜1 mL). Five fractions of EVs were collected in 0.5 mL fractions (approximately 8 drops off the column per fraction). The column was regenerated with three sequential 1 mL washes with regeneration buffer and stored at 4° C. in storage buffer (50% glycerol, 0.02% sodium azide in TBS).

Cas9 in vitro cleavage assays. EVs were produced as described above with components transiently transfected in 10 cm dishes with the following DNA ratios: 6 μg scFv, 9 μg Cas9 vector, 5 μg sgRNA vector, and 1 μg mTFP1 transfection control. EVs were lysed by incubation with mammalian protein extraction reagent (MPER, Thermo Fisher) for 10 min at room temperature (20-23° C.) with gentle agitation. 200 ng of linearized target plasmid template was added to vesicles with Cas9 reagent buffer (IDT, Alt-R CRISPR-Cas9 System), and samples were incubated at 37° C. for 1 h. Proteinase K (Thermo Fisher) was added to samples at 1 μL per 10 μL of reaction mixture and incubated at 55° C. for 10 min. Samples were run on a 1% agarose gel stained with SYBR safe (Thermo Fisher) and imaged using a BioDoc-It imaging system (VWR).

Primary CD4⁺ T cell isolation, culture, and activation. Leukocyte Reduction System (LRS) cones (StemCell Technologies 200-0093) from different donors were obtained and processed within 24 h of the initial blood draw. Peripheral blood mononuclear cells (PBMCs) were isolated by Ficoll gradient centrifugation. Briefly, fresh blood was mixed in a 1:1 ratio with Ca2+ and Mg2+ free Hank's Balanced Salt Solution (HBSS). Buffy coats were diluted in a 1:10 ratio with HBSS. 30 mL of the respective HBSS/blood solution were transferred to 50 mL Falcon tubes and underlaid with 12 mL Ficoll-Paque PLUS (Amersham/GE healthcare). After density gradient centrifugation (1,000 g, 20 min, no brakes) the PBMC layer was carefully removed, and the cells were washed twice with Ca2+ and Mg2+ free HBSS. CD4+ T cells were enriched with the Easysep Human CD4⁺ T-cell enrichment kit (Stemcell Technologies). Isolated CD4⁺ T cells were suspended in complete Roswell Park Memorial Institute (RPMI), consisting of RPMI-1640 (UCSF Cell Culture Facility (CCF)) supplemented with 5 mM 4-(2-hydroxyethyl)-1-piperazineethanesulfonic acid (HEPES, UCSF CCF), 2 mM Glutamine (UCSF CCF), 50 μg/mL penicillin/streptomycin (P/S, UCSF CCF), 5 mM nonessential amino acids (UCSF CCF), 5 mM sodium pyruvate (UCSF CCF), and 10% fetal bovine serum (FBS, Atlanta Biologicals). These cells were immediately stimulated on anti-CD3 coated plates (coated overnight with 10 μg/mL αCD3 (UCHT1, Tonbo Biosciences)) in the presence of 5 μg/mL soluble anti-CD28 (CD28.2, Tonbo Biosciences).

Statistical analysis. Statistical details are described in the figure legends. Unless otherwise stated, three independent biological replicates (cells) or technical replicates (beads) were analyzed per condition, and the mean fluorescence intensity of approximately 10,000 live single cells or beads were analyzed per sample. Unless otherwise indicated, error bars represent the standard error of the mean. Pairwise comparisons were made using two-tailed Student's t-tests in Excel with the null hypothesis that the two samples were equal. The significance threshold was set to 0.05. Tests were followed by a Benjamini-Hochberg procedure applied within each panel of a given figure to decrease the false discovery rate.

### Example 2: Introduction

Engineering T cells is an area of active investigation for the development of therapeutics to treat cancer, autoimmunity, and infectious disease.¹ One application of interest is treating HIV-infections using gene editing tools, such as CRISPR/Cas9, to eliminate the latent viral reservoir and functionally cure the disease. In vitro delivery of Cas9 with single guide RNA (sgRNA) targeting HIV sequences can ablate HIV proviruses in latently infected cells, and viral suppression is enhanced by using sgRNA targeting different locations or conserved regions in the HIV provirus. However, translation of this strategy remains difficult due to the challenges associated with in vivo delivery of Cas9. A promising approach is use of adeno-associated virus (AAV) gene delivery vehicles, but safety and efficacy are still somewhat limited by anti-vector immunity and limited tissue tropism. An exciting frontier is using virus-like particles (VLPs) to deliver Cas9 nucleases or base editors although it remains unclear whether the immunogenicity of viral proteins will limit these approaches.¹³ Synthetic nanoparticle-nucleic acid (e.g., mRNA) delivery is a promising alternative to viral vectors, and this approach achieved in vivo delivery of mRNA conferring sustained expression of chimeric antigen receptors in murine T cells. However, achieving efficient and specific targeting in a manner that confers the transient expression of Cas9 needed to avoid off-target effects remains challenging. These general difficulties are uniquely compounded by the challenge of delivering any cargo to T cells, which exhibit low rates of endocytosis. Altogether, there exists substantial opportunity to improve delivery systems that could enable delivery of biologics to T cells inside a patient.

A promising emerging strategy is the use of extracellular vesicles (EVs) to deliver biomolecular cargo. EVs are nanoscale, membrane-enclosed particles secreted by all cells that naturally encapsulate proteins and nucleic acids during biogenesis. EVs mediate intercellular communication, delivering their contents to recipient cells to affect cellular function.^{18,19} Intrinsic properties such as non-toxicity and non-immunogenicity,^{20,21} as well as the ability to engineer surface and luminal cargo loading, make EVs an attractive platform for delivering a wide range of therapeutics. Cargo can be incorporated into vesicles either by overexpression in the producer cells and subsequent loading during EV generation or by chemically modifying vesicles post-biogenesis.^{20,22} While downstream modification of EVs may have advantages in terms of cargo loading flexibility, this approach requires more extensive purification and introduces challenges from a manufacturing and regulatory standpoint.

Several recent studies have investigated the use of EVs to deliver Cas9 for treatment of cancer, hepatitis B, and genetic diseases, highlighting the promise of this method for achieving intracellular Cas9 delivery.²³⁻²⁵ However, many exploratory studies have employed EV engineering methods known to introduce artifacts in downstream experiments, which obscures how functional effects may be attributable to EVs. Of particular concern, transfecting EV producer cells with lipoplexes, loading EVs with electroporation methods known to result in cargo aggregation, or isolating EVs with commercial kits not intended for functional delivery applications have been shown to introduce artifacts.²⁶⁻²⁸ Exploration of EVs for treating HIV is also an area of active investigation, through approaches such as Cas9-mediated excision of proviruses in microglial cells,²⁹ repressing viral replication with zinc finger-fused methyltransferases,³⁰ or killing of infected cells using HIV Env-targeted vesicles,³¹ but these fundamental demonstrations have not yet been developed into methods for achieving specific delivery and treatment of T cells using a viable, translatable approach.

Here, this need was addressed by developing an integrated bioengineering strategy for genetically engineering the self-assembly of multifunctional EVs.

### Example 3: Assessing Engineered EVs

To promote specific interactions between EVs and target cells and facilitate EV uptake, a promising strategy is displaying targeting moieties on the EV surface. This strategy was pioneered using display of small peptides,^{20,22} although we and others have demonstrated that these effects are modest and variable.³⁴ Recently, display of high affinity targeting domains, including nanobodies and antibody single chain variable fragments (scFvs), conferred EV targeting to receptors such as EGFR and HER2.³⁵⁻³⁷ In these reported cases, scFv display was achieved by fusion to the C1C2 lactadherin domain, which binds to phosphatidylserine on the outer membrane leaflet of some EVs.

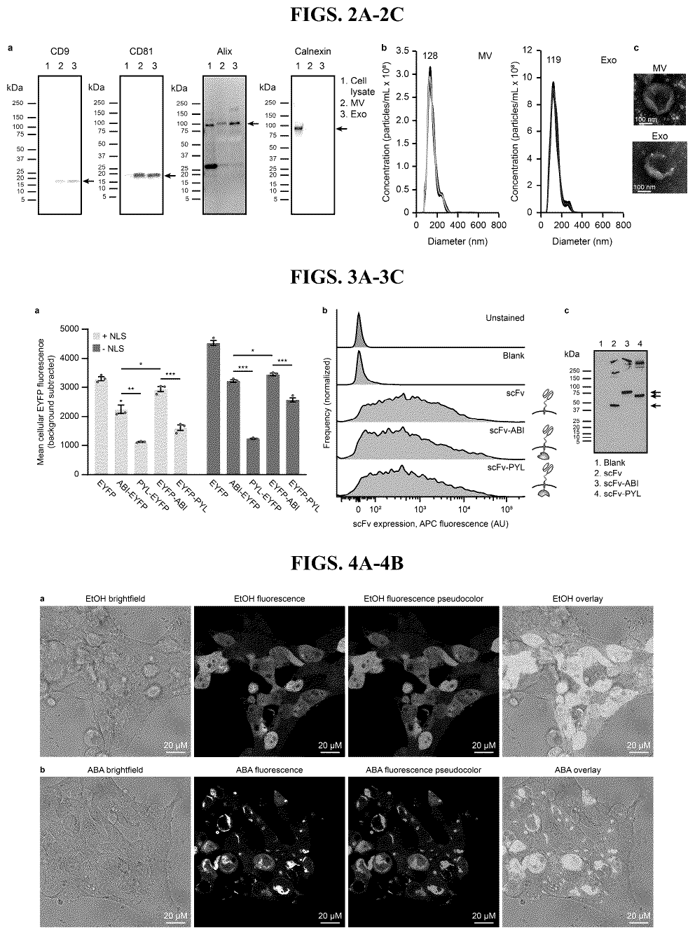

To test engineered EVs, two vesicle populations were isolated using a previously validated differential centrifugation method.^{41,42} EVs are best defined by the separation method used for their isolation;²⁸ for convenience, hereafter the fraction isolated at 15,000×g is termed “microvesicles” (MV) and the fraction isolated at 120,416×g is termed “exosomes” (exo). Vesicles were enriched in canonical markers such as CD9, CD81, and Alix and depleted in the endoplasmic reticulum protein calnexin (FIG. 2A). Both populations comprised vesicles averaging ˜120-140 nm in diameter and exhibited the expected “cup shaped” morphology (FIGS. 2B-2C).

### Example 4: Abscisic Acid-Inducible Dimerization Domains Enable an Active EV Cargo Loading System

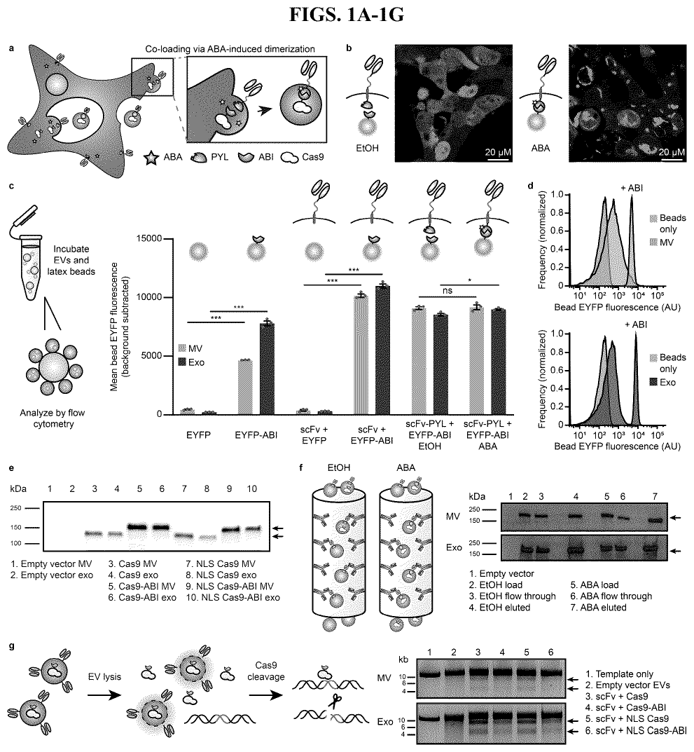

We next sought to further engineer EVs to load a therapeutic cargo of interest. Overexpression of cytosolic cargo in EV producer cells results in passive loading into vesicles during biogenesis via mass action.⁴⁶ Increasing cargo content in EVs would potentially produce a more potent delivery vehicle. In order to both enhance cargo protein loading and increase the likelihood that a given vesicle will incorporate both a cytosolic cargo protein and our membrane-bound scFv, we designed a small entity dimerization-based loading system (FIG. 1A). Systems using light or small entities (e.g., rapamycin) as inducers have been reported to aid EV cargo loading,^{47,48} but light is difficult to scale to large volumes and rapamycin-induced dimerization is so tight that it is functionally irreversible.⁴⁹ Therefore, we explored a new strategy based upon the plant hormone abscisic acid (ABA)-inducible interaction between truncated versions of the abscisic acid insensitive 1 (ABI) and pyrabactin resistance-like (PYL) proteins.⁵⁰ This “ABA” system confers several advantages: association is rapid; the dimerization is reversible, presumably allowing for cargo release in recipient cells; ABA is inexpensive and non-toxic; and small molecule-regulated loading is more readily applicable to biomanufacturing than is control by light. We first investigated fusing the ABI and PYL domains to the luminal side of our scFv construct and to either the 5′ or 3′ end of a cytosolic or nuclear-localized EYFP cargo protein to determine effects on protein expression and function. Fusion with the PYL domain reduced expression (or destabilized) EYFP (FIG. 3A), while the scFv was tolerant to fusions with either ABI or PYL domains (FIGS. 3B-3C). Thus, we moved forward with the scFv-PYL and EYFP-ABI (3′ fusion) constructs. ABA-induced dimerization of ABI and PYL in this setup was readily evident by microscopy (FIG. 1B and FIGS. 4A-4B).

### Example 5: The ABI Domain Alone Drives Protein Incorporation into EVs

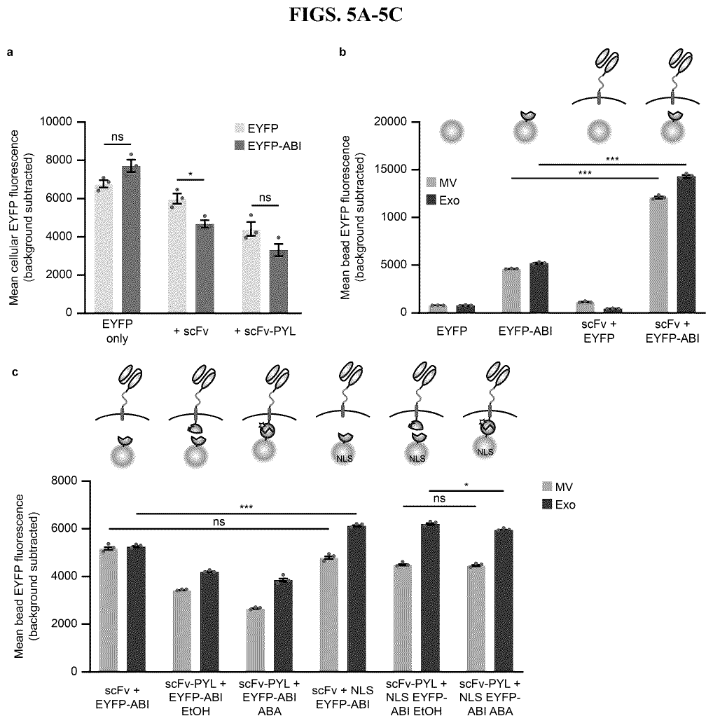

To investigate cargo protein loading, vesicles were adsorbed to latex beads and analyzed by flow cytometry. Surprisingly, no increase in EV loading was observed with ABA treatment, and across all conditions, constructs containing the ABI domain demonstrated a higher degree of loading than did those lacking this domain (FIGS. 1C-1D). This effect was not attributable to ABI-dependent increases of protein expression in producer cells (FIG. 5A). ABI-enhanced loading was evident when paired with the scFv alone or the scFv-PYL construct, indicating that intrinsic ABI-enhanced loading is independent of ABI-PYL interactions (FIG. 1C). The presence of the scFv conferred an added benefit in protein loading over an EYFP-ABI only control, for unknown reasons (FIG. 5B). In order to investigate the role of subcellular localization on the EV loading process, we introduced a nuclear localization sequence (NLS) to EYFP-ABI and compared loading to the purely cytosolic construct. ABA-induced dimerization again had a negligible effect on cargo loading, and addition of an NLS to EYFP-ABI did not diminish loading into EVs (FIG. 5C). Altogether, these data support the serendipitous discovery that ABI comprises a novel, potent EV cargo protein loading tag.

### Example 6: The ABI Domain Mediates Cas9 Loading into EVs

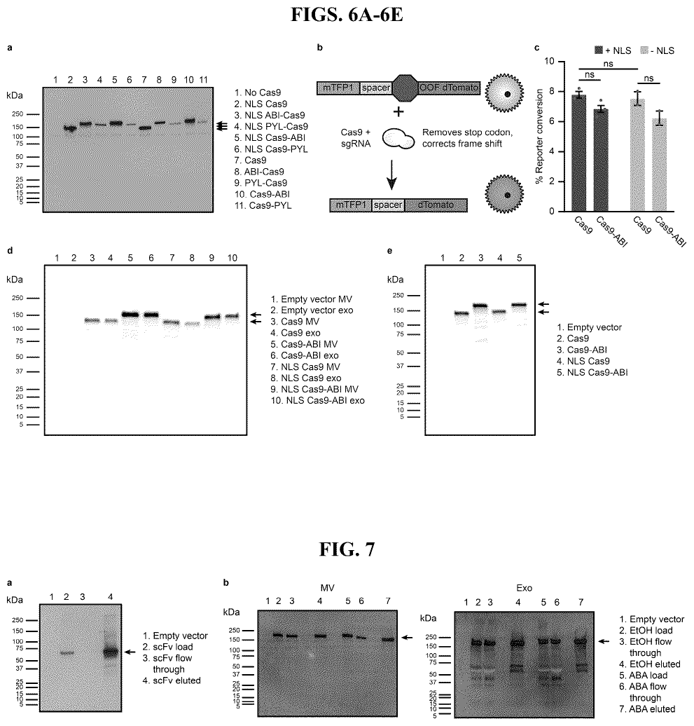

We next investigated whether ABI can be used to load EVs with functional cargo. As model, we selected S. Pyogenes Cas9 ribonucleoprotein complexes (RNPs)-RNPs can be synthesized in producer cells (and are thus consistent with the GEMINI strategy) and because RNPs must travel to the nucleus of recipient cells to act on genomic targets, this system comprises a stringent test for functional EV-mediated delivery. ABI was fused to the N- or C-terminus of Cas9, and in general, expression patterns matched those observed for EYFP (FIG. 6A). Thus, we moved forward with the Cas9-ABI (3′ fusion) constructs. We also investigated whether addition of an NLS or ABI domain impacted Cas9 function. When expressed via transfection (along with a cognate sgRNA) in reporter Jurkat T cells, Cas9 fusion constructs exhibited similar nuclease activity (FIGS. 6B-6C). When Cas9 constructs were expressed in producer cells, the NLS minimally influenced Cas9 loading into EVs, while the ABI domain noticeably increased Cas9 loading (FIG. 1E and FIG. 6D) but not overall expression in producer cells (FIG. 6E). These trends are consistent with those observed with EYFP and demonstrate the utility of the ABI loading tag across multiple cargo proteins.

### Example 7: Membrane scFvs and ABI-Fused Cas9 Co-Load into EVs

An important, but largely unexplored, factor to consider in engineering EV-based therapeutics is the extent to which multiple cargo types localize to the same vesicles in a population. Although ABI (alone) successfully loads protein into EVs, it remained unknown whether dimerization of cargo and display proteins could enhance co-loading into EVs (i.e., co-loading of both the scFv and Cas9 into individual vesicles). To evaluate this question, we generated vesicles from cells expressing scFv-PYL and Cas9-ABI treated with ABA or a vehicle control and isolated scFv-displaying vesicles via the 3×FLAG tag located on the N-terminus of the scFvs by affinity chromatography (FIG. 7A).⁵¹ High levels of Cas9 were found in scFv-enriched vesicles, independent of ABA treatment, indicating that ABI-tagging of cargo is sufficient to achieve substantial scFv and Cas9 co-localization in EVs (FIG. 1F and FIG. 7B).

### Example 8: EV-Loaded Cas9 Exhibits Nuclease Function

To evaluate whether EV-encapsulated Cas9 RNPs are functional, we developed a direct in vitro assay. EVs from Cas9 and sgRNA-expressing cells were lysed and incubated with a plasmid encoding the sgRNA target sequence (FIG. 1G). Plasmids treated with lysed RNP-containing EVs showed the expected specific cleavage products under all conditions tested. The presence or absence of an NLS did not impact cleavage efficiency in this assay, but Cas9 fused to the ABI domain exhibited some reduced cleavage for both vesicle populations. This pattern contrasts with that observed in the transfection-based Cas9 assay (FIG. 6C), and thus it is not clear whether this partial effect (e.g., a potential reduction in Cas9 turnover rate) is meaningful in a cellular delivery context. Thus, both ABI+ and ABI− constructs were evaluated in subsequent experiments.

### Example 9: Discussions

The technology reported here involved the unexpected discovery that the ABI domain (from the ABA dimerization system) facilitates EV cytosolic cargo protein loading even without with ABA. The mechanism of this effect is unknown. ABI is not predicted by WoLF PSORT (genscript.com/wolf-psort.html) to localize to the cell membrane or endosomal pathways. An advantage of this system is that ABI-mediated loading is easier to implement than multi-domain dimerization systems (using light,⁴⁷ rapamycin,⁴⁸ or Dmr domains²⁹) or tags that require overexpression of helper proteins to facilitate trafficking into vesicles, such as the WW domain and Ndfip 1.⁶⁶ Other active loading tags have recently been explored by Codiak Biosciences,⁶⁷ in this case deriving a tag from a membrane-associating protein, though the reversibility of such interactions has yet to be established. Although increased Cas9 loading did not confer additional DNA cleavage in our in vitro assay, potentially because this particular Cas9 fusion strategy reduced Cas9 turnover rate (FIG. 6C), higher cargo loading is likely beneficial in cell delivery contexts where EVs must overcome additional barriers of uptake, fusion, cytosolic release, and intracellular trafficking. In such contexts, the advantage of a higher dose with more shots on goal may outweigh slower reaction rates. It is also possible that the ABI fusion strategy may be refined in future work to mitigate any effects on Cas9 activity.

A notable feature of this study was the selection of methods that avoid artifacts found in EV studies. One general and often overlooked artifact with EV functional delivery experiments is the risk of transfer of residual producer cell transfection reagent; particles from cells transfected with lipoplexes can mediate functional effects erroneously attributed to EVs.²⁶ We minimized such risks by employing a transfection method that is unlikely to transfer plasmids to T cells.

### Example 10: Prophetic Functional Delivery of Cas9 to T Cells

A further contemplated example includes using our active loading strategy to deliver Cas9-sgRNA complexes to recipient cells to mediate gene editing. An example of this strategy would be to express Cas9-ABI (fused using our technology in one of the implementations contemplated here) and an anti-CXCR4-targeting sgRNA in HEK293FT cells; to harvest the EVs produced from these cells using standard methods; to deliver these EVs to T cells (e.g., Jurkat T cells or primary human T cells); and after some time, to evaluate whether the CXCR4 locus has been cut and repaired in these recipient T cells (e.g., using high throughput sequencing).

The technologies employed here are generalizable and amenable to large scale production and biomanufacturing. Our strategy of genetically programming the self-assembly of multifunctional particles avoids the need for post-harvest chemical modification that necessitates further purification, lower EV yields, and may incur regulatory challenges. Although we used transient transfections for some transgenes, such genes are regularly expressed from inducible promoters for production of biologics. We anticipate that the integrated tools developed here for lipid bilayer particle (e.g., CDMP) cargo loading, and vesicle fusion will be widely applicable for a range of applications and targets, providing a flexible platform for engineering lipid bilayer particle (e.g., CDMP) therapeutics.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A chimeric protein or peptide comprising a cargo-loading domain comprising an abscisic acid-insensitive 1 (ABI1) sequence linked directly or indirectly to a cargo entity.

2. A chimeric protein or peptide comprising:
(a) a cargo entity; and
(b) a cargo-loading domain comprising an abscisic acid-insensitive 1 (ABI1) sequence, wherein the cargo entity and cargo-loading molecule are linked directly or indirectly.

3. The chimeric protein or peptide of claim 1, wherein the linker comprises:
(1) an amino acid sequence selected from SEQ ID NO: 10 (TSGGGGSGGGSGGGS), SEQ ID NO: 12 (TRGGGGSGGGSGGGS), SEQ ID NO: 14 (GGGGSGGGSGGGSTG), SEQ ID NO: 15 (DQSNSEEAKKEEAKKEEAKKSNS), SEQ ID NO: 16 (SGGGSGGGSGGGSGGSGGSGGGSGGSGGSGGGSGGGSGGG), and SEQ ID NO: 17 (ESKYGPPAPPAP); or
(2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 10, 12, 14, 15, 16, or 17.

4. The chimeric protein or peptide of claim 1, wherein the cargo-loading domain is a truncated variant of a wild-type protein that comprises an extracellular vesicle targeting domain.

5. The chimeric protein or peptide of claim 1, wherein the cargo-loading domain comprises residues 126-423 of wild type ABI1.

6. The chimeric protein or peptide of claim 1, wherein the cargo-loading domain comprises:
(SEQ ID NO: 6)
MTRVPLYGFTSICGRRPEMEAAVSTIPRFLQSSSGSMLDGRFDPQSAAH
FFGVYDGHGGSQVANYCRERMHLALAEEIAKEKPMLCDGDTWLEKWKKA
LFNSFLRVDSEIESVAPETVGSTSVVAVVFPSHIFVANCGDSRAVLCRG
KTALPLSVDHKPDREDEAARIEAAGGKVIQWNGARVFGVLAMSRSIGDR
YLKPSIIPDPEVTAVKRVKEDDCLILASDGVWDVMTDEEACEMARKRIL
LWHKKNAVAGDASLLADERRKEGKDPAAMSAAEYLSKLAIQRGSKDNIS
VVVVDLK,
(SEQ ID NO: 7)
VPLYGFTSICGRRPEMEAAVSTIPRFLQSSSGSMLDGRFDPQSAAHFFG
VYDGHGGSQVANYCRERMHLALAEEIAKEKPMLCDGDTWLEKWKKALFN
SFLRVDSEIESVAPETVGSTSVVAVVFPSHIFVANCGDSRAVLCRGKTA
LPLSVDHKPDREDEAARIEAAGGKVIQWNGARVFGVLAMSRSIGDRYLK
PSIIPDPEVTAVKRVKEDDCLILASDGVWDVMTDEEACEMARKRILLWH
KKNAVAGDASLLADERRKEGKDPAAMSAAEYLSKLAIQRGSKDNISVVV
VDLK,
a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 6 or 7, or
a functional fragment of SEQ ID NO: 6, SEQ ID NO: 7, or a variant amino acid sequence thereof.

7. The chimeric protein or peptide of claim 1, wherein the cargo entity is a cytosolic cargo entity.

8. A lipid bilayer particle loading system comprising the chimeric protein or peptide of claim 1 and a second chimeric protein or peptide comprising (i) a second cargo molecule, and (ii) and membrane-bound domain comprising an abscisic acid (ABA)-binding sequence, wherein the second chimeric protein or peptide optionally comprises a second linker that connects the second cargo entity and the ABA-binding sequence.

9. The particle loading system of claim 8, wherein the ABA-binding sequence comprises a pyrabactin resistance 1-like (PYL1) sequence.

10. The particle loading system of claim 9, wherein the PYL1 sequence comprises residues 33-209 of wild type PYL1.

11. The particle loading system of claim 9, wherein the PYL1 sequence comprises
(SEQ ID NO: 2)
MGGGAPTQDEFTQLSQSIAEFHTYQLGNGRCSSLLAQRIHAPPETVWSV
VRRFDRPQIYKHFIKSCNVSEDFEMRVGCTRDVNVISGLPANTSRERLD
LLDDDRRVTGFSITGGEHRLRNYKSVTTVHRFEKEEEEERIWTVVLESY
VVDVPEGNSEEDTRLFADTVIRLNLQKLASITEAMN,
(SEQ ID NO: 3)
TQDEFTQLSQSIAEFHTYQLGNGRCSSLLAQRIHAPPETVWSVVRRFDR
PQIYKHFIKSCNVSEDFEMRVGCTRDVNVISGLPANTSRERLDLLDDDR
RVTGFSITGGEHRLRNYKSVTTVHRFEKEEEEERIWTVVLESYVVDVPE
GNSEEDTRLFADTVIRLNLQKLASITEAMN,
or
a variant amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 2 or 3, or
a functional fragment of SEQ ID NO: 6, SEQ ID NO: 7, or a variant amino acid sequence thereof.

12. The particle loading system of claim 8, wherein the second linker comprises:
(1) an amino acid sequence selected from SEQ ID NO: 10, SEQ ID NO: 12, SEQ ID NO: 14, SEQ ID NO: 15, SEQ ID NO: 16, and SEQ ID NO: 17; or
(2) an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of SEQ ID NOs: 10, 12, 14, 15, 16, or 17.

13. The particle loading system of claim 8 further comprising abscisic acid (ABA).

14. The particle loading system of claim 8, wherein the second cargo entity is a membrane-bound cargo molecule, wherein the cargo entity optionally comprises (i) a targeting protein and (ii) a transmembrane domain, and wherein the targeting protein is selected from an antibody, a Fab, a Fab′, a F(ab′)₂, a Fd, a scFv, a single-chain antibody, a disulfide-linked Fvs (sdFv), a de novo-designed binding molecule, an affinibody, a DARPIN, and a nanobody.

15. A lipid bilayer particle comprising the chimeric protein or peptide claim 1; wherein the lipid bilayer particle is a CDMP.

16. The particle of claim 15, wherein the lipid bilayer particle is engineered.

17. The particle of claim 15, wherein the CDMP is selected from the group consisting of an extracellular vesicle, virus particles, virus-like particles (VLPs), apoptotic bodies, platelet-like particles, and a combination thereof.

18. The particle of claim 15, wherein the CDMPs are extracellular vesicles selected from the group consisting of exosomes, microvesicles, and combinations thereof.

19. A nucleic acid encoding the chimeric protein or peptide of claim 1.

20. The nucleic acid of claim 19, wherein the cargo-loading domain of the chimeric protein or peptide is encoded by
(SEQ ID NO: 5)
ATGACCAGAGTGCCCCTGTACGGCTTCACCAGCATTTGTGGCAGACGGC
CCGAAATGGAAGCCGCCGTGTCTACAATCCCCAGATTCCTCCAGAGCAG
CAGCGGCTCCATGCTGGACGGCAGATTCGATCCTCAGAGCGCCGCTCAC
TTCTTCGGCGTGTACGATGGACATGGCGGAAGCCAGGTGGCCAACTACT
GCCGCGAAAGAATGCATCTGGCCCTGGCCGAGGAAATCGCCAAAGAAAA
GCCCATGCTGTGCGACGGCGACACCTGGCTGGAAAAGTGGAAGAAGGCC
CTGTTCAACAGCTTCCTGAGAGTGGACAGCGAGATCGAGAGCGTGGCCC
CTGAAACAGTGGGCAGCACATCTGTGGTGGCCGTGGTGTTTCCCAGCCA
CATCTTCGTGGCTAACTGCGGCGATAGCAGAGCCGTGCTGTGCAGAGGA
AAAACAGCCCTGCCTCTGTCCGTGGACCACAAGCCTGATAGAGAGGATG
AGGCCGCCAGAATTGAAGCCGCTGGCGGCAAAGTGATCCAGTGGAATGG
CGCTAGAGTGTTCGGCGTGCTGGCCATGAGTAGATCCATCGGCGATAGA
TACCTGAAGCCTAGCATCATCCCCGATCCTGAAGTGACCGCCGTGAAGA
GAGTGAAAGAGGACGACTGCCTGATCCTGGCCTCTGACGGTGTCTGGGA
CGTGATGACAGATGAAGAGGCCTGCGAGATGGCCCGGAAGAGAATCCTG
CTGTGGCACAAGAAAAACGCCGTGGCCGGGGATGCTTCTCTGCTGGCTG
ACGAGAGAAGAAAAGAGGGCAAAGACCCCGCTGCCATGTCTGCCGCCGA
GTACCTGTCTAAGCTGGCCATCCAGAGAGGCAGCAAGGACAACATCAGC
GTGGTGGTCGTGGACCTGAAA,
a variant nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 5, or
a functional fragment of SEQ ID NO: 5 or a variant nucleic acid sequence thereof.

21. The nucleic acid of claim 19, wherein the ABA-binding sequence of the second chimeric protein or peptide is encoded by
(SEQ ID NO: 1)
ATGGGCGGAGGAGCCCCTACCCAGGACGAGTTCACCCAGCTGAGCCAGA
GCATCGCTGAGTTCCACACCTACCAGCTGGGAAACGGACGCTGTTCCAG
CCTGCTGGCACAGAGAATCCACGCTCCTCCTGAGACAGTGTGGAGTGTG
GTGCGCAGATTCGACCGCCCTCAGATTTACAAGCACTTCATCAAGAGCT
GCAACGTGAGCGAGGACTTCGAGATGAGAGTGGGATGTACCAGAGATGT
GAACGTGATCAGCGGACTGCCTGCCAACACCAGCAGAGAGAGACTGGAC
CTGCTGGACGATGACCGCAGAGTGACCGGCTTCAGCATCACCGGAGGTG
AGCACAGACTGAGAAACTACAAGAGCGTGACCACCGTCCACCGCTTCGA
GAAGGAAGAGGAAGAGGAGCGCATCTGGACCGTGGTGCTGGAGAGCTAC
GTCGTGGACGTGCCCGAGGGCAACAGCGAAGAGGATACCCGCCTGTTCG
CTGACACCGTGATCAGACTGAACCTCCAGAAGCTGGCCAGCATCACCGA
GGCAATGAAC,
a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO: 1, or
a functional fragment of SEQ ID NO: 1 or a variant nucleic acid sequence thereof.

22. The nucleic acid of claim 19, wherein the linker and/or second linker are encoded by one of SEQ ID NOs: 9 (ACTAGTGGCGGCGGAGGCAGCGGAGGCGGATCTGGCGGAGGATCT), 11 (ACGCGTGGCGGCGGAGGCAGCGGAGGCGGATCTGGCGGAGGATCT), or 13 (GGCGGCGGAGGAAGTGGCGGCGGATCTGGCGGAGGATCTACCGGT),
or a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to one of SEQ ID NOs: 9, 11, or 13.

23. A cell comprising the chimeric protein or peptide of claim 1.

24. The cell of claim 23, wherein the cell is a mammalian cell, wherein the mammalian cell is optionally selected from HEK293, HEK293FT, a mesenchymal stem cell, a megakaryocyte, an induced pluripotent stem cell (iPSC), a T cell, an erythrocyte, an erythropoetic precursor, and an iPSC-derived version of any of the preceding cells.

25. A method of loading a cargo entity into lipid bilayer particles, comprising expressing in a cell the chimeric protein or peptide of claim 1.

26. The method of claim 25, wherein loading of the cargo entity of the chimeric protein or peptide is enhanced compared to passive cargo loading.

27. The method of claim 25, wherein the cargo entity is a viral nucleocapsid, a synthetic nucleic acid, a transcription factor, a recombinase, a base editor, a prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, or a ribonucleoprotein complex.

28. A method of loading two cargo entities into cell-derived membrane particle, comprising expressing in a cell the lipid bilayer particle loading system of claim 8.

29. The method of claim 28, wherein co-localization of the cargo entity of the chimeric protein or peptide and the second cargo entity of the second chimeric protein or peptide is enhanced compared to passive cargo loading.

30. The method of claim 28, wherein the cargo entity is a viral nucleocapsid, a synthetic nucleic acid, a transcription factor, a recombinase, a base editor, a prime editor, a nuclease (e.g., a TALEN, ZFN, etc.), a kinase, a kinase inhibitor, an activator or inhibitor of receptor-signaling, an intrabody, a chromatin-modifying synthetic transcription factor, a natural transcription factor, a CRISPR-Cas family protein, a DNA molecule, an RNA molecule, or a ribonucleoprotein complex.
